(19) United States
(12) Patent Application Publication
(10) Pub. No.: US 2008/0219928 A1
(43) Pub. Date: Sep. 11, 2008

(54) Measuring Gastrointestinal Parameters

(76) Inventors: Jane A. Becker, Fort Wright, KY (US); Alex Chun Ching Lin, Cincinnati, OH (US); Gregory P. Drach, Liberty Township, OH (US); Wei-Kuo Chang, Taipei City (TW)

Correspondence Address: WHYTE HIRSCHBOECK DUDEK S C, 555 EAST WELLS STREET, SUITE 1900, MILWAUKEE, WI, 53202, US

(21) Appl. No.: 12/019,754

(22) Filed: Jan. 25, 2008

Related U.S. Application Data
(63) Continuation-in-part of application No. 10/787,705, filed on Feb. 26, 2004.
(60) Provisional application No. 60/450,551, filed on Feb. 26, 2003.
(60) Provisional application No. 60/526,345, filed on Dec. 1, 2003.

(51) Int. Cl. A61K 49/00

(52) U.S. Cl. 424/9.1

(57) ABSTRACT

The present application relates to methods for determining gastric residual volumes and amounts of dietary formula in gastric contents using measurements of soluble solids concentrations in gastric contents, and in some embodiments, a concentrate having a relatively high concentration of soluble solids. The methods can be used for measuring gastric residual volumes of subjects who have fasted or have a low or reduced gastric content.

### PRIORITY CLAIM AND RELATED APPLICATION

The present application is a continuation-in-part of U.S. patent application Ser. No. 10/787,705, filed Feb. 26, 2004, which claims priority from U.S. Provisional Application entitled “Monitoring Gastric Residual Volume and Formula Concentration”, Ser. No. 60/450,551, filed Feb. 26, 2003 and U.S. Provisional Application entitled “Measuring Gastrointestinal Parameters”, Ser. No. 60/526,345, filed Dec. 1, 2003, which are incorporated herein by reference in their entirety for all purposes.

### FIELD

The present application relates to methods of measuring gastric residual volumes, for monitoring emptying and evaluating feeding tolerance in a subject or patient receiving enteral nutrition.

### BACKGROUND

Enteral nutrition is generally preferred over parenteral nutrition because of its lower cost, lower rate of complications, and effective preservation of gut structure and function. Many critically ill patients cannot tolerate nasogastric tube feeding, developing manifestations of intolerance including nausea, vomiting, abdominal distension, and aspiration. Gastric residual volumes are widely used to evaluate feeding tolerance and gastric emptying. High gastric residual volumes raise concern about intolerance to gastric feeding and the potential risk for regurgitation and aspiration pneumonia. Values of gastric residual volumes cited as being high in patients receiving nasogastric feeding typically range from 75 to 500 ml. However, controversy exists regarding the accuracy of these measurements.

Conventional use of gastric residual volume obtained by aspiration via a syringe is often inaccurate and unreliable in measuring true volume of contents present in the stomach at any given time. Although gastric residual volumes obtained by aspiration from a nasogastric feeding tube (Asp GRVs) are widely used to evaluate tolerance and gastric emptying of enteral feedings, several reports have shown that Asp GRVs by themselves are poorly correlated with gastric emptying, incidence of regurgitation, and risk of pulmonary aspiration. The conventional practice of calculating gastric residual volume typically does not take into account the fact that fluids accumulating in the stomach of the patient during nasogastric tube feeding often include not only the tube feeding formula itself, but also swallowed saliva and gastric secretions. Therefore, gastric residual volumes alone cannot distinguish the additional volume of endogenous secretions in a patient who is effectively emptying the volume of exogenous feeding. As a result, use of aspirated gastric residual volumes as a monitor for gastric emptying is currently limited by poor sensitivity and an inability to aspirate the complete volume of gastric contents. The sensitivity of Asp GRVs for detecting aspiration through a range of designated threshold values is only 1.9-8.1%. Moreover, conventional calculation of aspirated gastric residual volumes is imprecise and cannot distinguish the components of retained enteral formula from the large volume of naturally occurring endogenous secretions.

The true gastric residual volume is determined by the dynamic balance between input (e.g., infused formula and endogenous secretions) and output (e.g., gastric emptying of the stomach). Typically, endogenous secretions contribute 1500 ml of salivary secretions and 3000 ml of gastric juice per day. Therefore, an accurate method to determine total volume of contents in the stomach and a system to differentiate the exact volume of the component of formula in that mixed solution of gastric contents would be helpful in evaluating treatment in patients receiving gastric feeding. Recent evidence suggests that conventional calculation of Asp GRVs (especially when performed by syringe aspiration) does not adequately measure true volume of gastric contents remaining in the stomach of Asp because it cannot differentiate the components of the gastric contents (infused formula versus endogenous secretions).

Refractometry is a useful technique because of its minimal expense and ease of utilization. The Brix value is a measurement, typically measured using refractometry, of total soluble solids in solution. This value is a constant for a pure substance under standard conditions of temperature and pressure. The Brix value closely correlates with the molar fractions of the components. In other words, the overall Brix value of a mixed solution approximates the additive sum of the Brix values of its individual components. Brix values have been used in a number of clinical settings to determine the concentration of mixed substances such as drugs, food, fruit juices, and parenteral nutrition solutions. However, there is not much known about Brix values related to enteral nutrition solutions or its correlation to gastric emptying. For example, it is currently unknown what nutrients in dietary formula are necessary for Brix value determination, what parameters affect Brix value measurements of gastric contents or the correlation between Brix values and gastric content components.

### SUMMARY

A method of monitoring feeding tolerance in a patient receiving enteral nutrition is described and involves infusing dietary formula into the stomach of a patient, measuring the aspirated gastric residual volume of the infused patient, evaluating the aspirated gastric residual volume, whereby a lower gastric residual volume value indicates acceptable feeding tolerance and a higher gastric residual volume value indicates additional monitoring, calculating a Brix value ratio obtained by a Brix value dilution test, evaluating the Brix value ratio, whereby a ratio of less than approximately 70% indicates acceptable feeding tolerance and a ratio above approximately 70% indicates additional monitoring, calculating the volume of formula remaining in the stomach, and evaluating the volume of formula remaining in the stomach, whereby a formula volume approximately less than the infusion rate indicates acceptable feeding tolerance and a formula volume greater than the infusion rate indicates additional monitoring.

A method of determining the concentration of dietary formula is also disclosed, involving infusing dietary formula to the stomach of a subject, measuring a Brix value of the infused dietary formula, determining a slope value derived from Brix values of serially diluted dietary formula over a determined concentration range, and dividing the Brix value of the infused dietary formula by the slope value.

A method is also provided for determining gastric residual volume by measuring the Brix value of a gastric content sample, adding a known volume of water to the gastric content sample to form a post-dilution sample, infusing the post-dilution sample from the stomach, measuring the Brix value of the aspirated post-dilution sample, and multiplying the Brix value of the gastric content sample by the known volume of water and dividing the resulting product by the difference between the pre-dilution Brix value and post-dilution Brix value.

Also methods of monitoring gastric content emptying and feeding tolerance in a patient receiving dietary formula, and methods of determining the volume of dietary formula volume remaining in the stomach and the volume of gastric juice in stomach are also disclosed.

These and other features will be appreciated from review of the following detailed description along with the accompanying figures.

### BRIEF DESCRIPTION OF THE DRAWINGS

FIG. 1 shows a representative determination of dietary formula concentration;

FIG. 2 shows a representative determination of gastric residual volume;

FIG. 3 shows a representative example of determining the volume of dietary formula remaining in stomach;

FIG. 4 shows a method of monitoring feeding tolerance in a patient receiving enteral nutrition using Brix value measurements in conjunction with conventional aspirated gastric residual volume measurements;

FIG. 5 shows an experimental derivation of calculating the gastric residual volume using Brix value measurements; and

FIG. 6 shows an derivation for calculating the gastric residual volume in a subject in accordance with another aspect of the invention.

### DETAILED DESCRIPTION

This present application describes the monitoring of gastric tolerance and gastric emptying through measurement of Brix values gastric contents. Method of

### Measuring the Concentration of Dietary Formula

Disclosed is a method of measuring the percent concentration of dietary formula by infusing dietary formula into the stomach of a subject. Infusing may be accomplished by nasogastric feeding, enteral nutrition feed, or any known tube feeding technique. Any dietary formula can be used with this method. For example, in one embodiment, the dietary formula is a dietary formula for gastric tube feeding or any type of enteral nutrition. In another embodiment, the dietary formula is liquid dietary formula or polymeric diet formula. Likewise, any ranges of the concentration of dietary formula can be monitored by this method. That is, 0-100% of the dietary formula can be monitored by refractometry. For example, full-strength polymeric diet would be considered 100% concentration dietary formula.

The Brix value of the infused dietary formula is measured. In one embodiment, the Brix value is measured with a refractometer. However, other known methods of obtaining a Brix value may also be used.

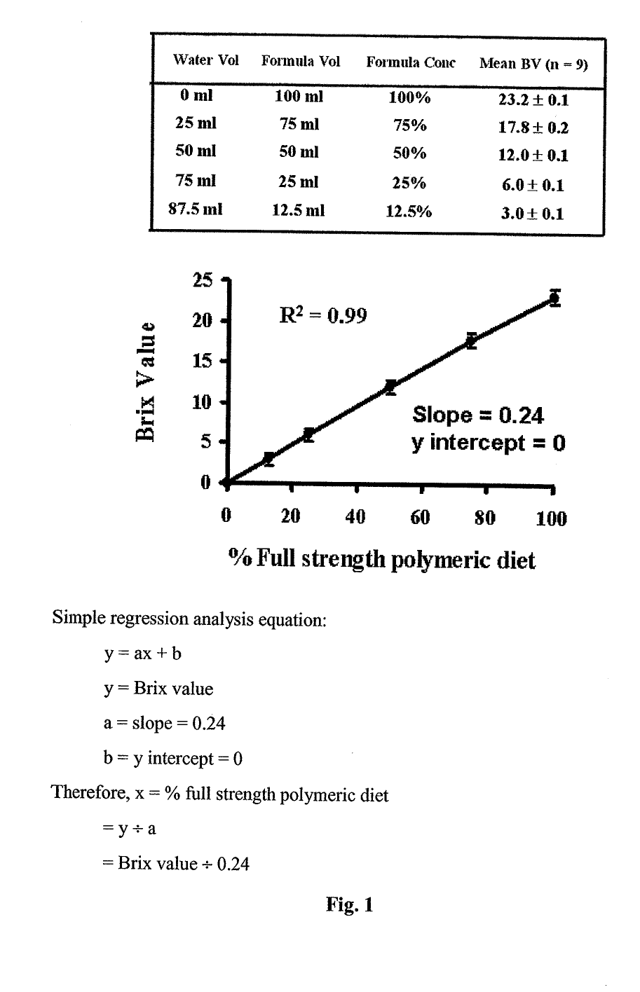

A slope value is derived from Brix values of serially diluted dietary formula over a determined concentration range. One embodiment is shown in FIG. 1. In this non-limiting example, the Brix values of an infused dietary formula are plotted against serial dilutions of that formula exhibited as concentration percentages of full strength (100%) dietary formula, or polymeric diet. The slope value is calculated according to known regression analysis methods. In some embodiments, the dietary formula may be diluted by distilled water, gastric juice, saliva, NaCl or dextrose solutions. However, other known diluents may also be used. In one embodiment, the slope value is 0.24. The percent concentration of dietary formula may be expressed as:

$$\% conc . dietary formula = \frac{Brix value infused dietary formula}{slope value}$$

The Brix values of polymeric dietary formula have a linear additive relationship with the dietary formula concentration (R²=0.99), which is exemplified in FIG. 1. With such a high degree of correlation, the measured Brix value may be correlated to the percent concentration of the formula (e.g., % full strength polymeric diet) at any dilution.

In other embodiments, the percent concentration of dietary formula in the gastric content is not affected by the body secretions, pH value, temperature or the residence time of the dietary formula in stomach. These and other features are further described in non-limiting detail through the examples provided herein.

### Method of Determining the Gastric Residual Volume

A method of determining gastric residual volume is also disclosed. The Brix value of a gastric content sample is measured to obtain a pre-dilution Brix value, BV1. The Brix value may be measured with a refractometer or any other device or technique known in the art. The gastric content sample may, for example, contain dietary formula, bodily secretions (e.g., gastric juices' or saliva), and mixtures thereof. Of course, other components known in the art may also be contained in the gastric content sample. The Brix value may be measured from a gastric content sample obtained in vitro or after the sample has been aspirated from the stomach of a patient.

A known volume of water sample is added to the gastric content sample to form a post-dilution sample. The amount of water added to the sample may be easily determined by one skilled in the art, depending on the size of the sample, the dietary formula, the condition of the subject, etc. In the case of a sample obtained in vivo, the water is added after the sample has been aspirated from the stomach of the patient. In some embodiments, the volume of water ranges between 10 ml to 500 ml. The gastric content is infused into the stomach using known infusion methods. Upon aspirating the resulting post-dilution sample from the stomach, the Brix value of the aspirated post-dilution sample is measured to obtain a value BV2. Calculation of the gastric residual volume may be expressed as:

$$\frac{BV 1 \times added volume of water}{( BV 1 - BV 2 )}$$

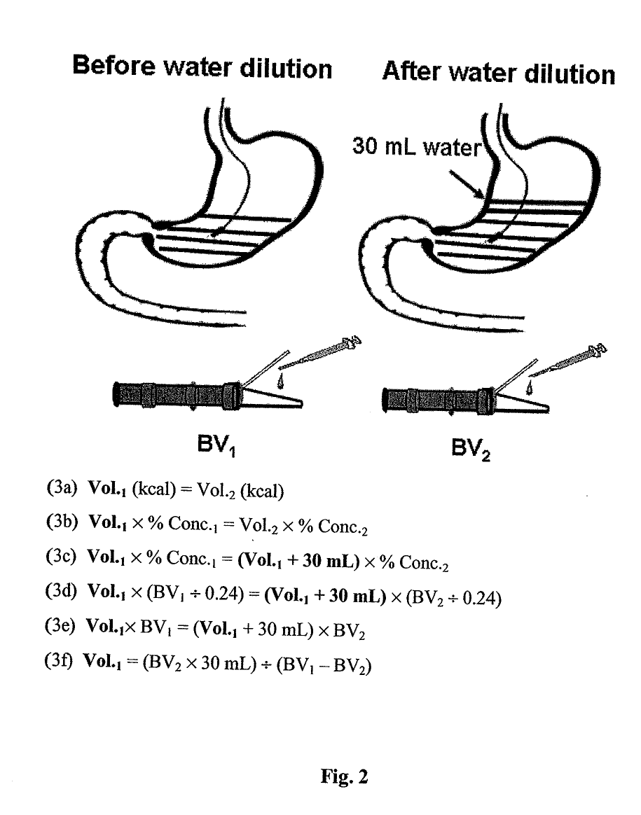

A representative determination of the gastric residual volume is exemplified in FIG. 2. Before water dilution, the calculated gastric residual volume was set as Vol. 1, % full strength polymeric dietary as % Conc. 1, and the Brix value of gastric contents as BV1. After 30 ml water dilution in the stomach, the gastric volume was set as Vol. 2, % full strength polymeric dietary as % Conc. 2, and the Brix value of gastric content as BV2. An added dilutional 30 ml volume of distilled water was infused via nasogastric tube. The stomach contents again were thoroughly mixed (using a 60-ml syringe, filled and emptied 3 times), and a 2 ml sample was obtained for a final Brix value measurement. The gastric residual volume (Vol. 1) was calculated according to the equation shown in FIG. 2.

### Method of Determining the Amount of Dietary Formula Volume Remaining and Gastric Juice Volume in Stomach

A method of determining the amount of dietary formula volume remaining in the stomach involves determining the gastric residual volume of a subject and multiplying this volume by the concentration of dietary formula. In some embodiments, this concentration may be expressed as the percentage full strength of dietary formula or polymeric diet.

The calculated gastric residual volume may be obtained from measuring the gastric residual volume. Any dietary formula can be used with this method. In one embodiment, the dietary formula is the dietary formula for gastric tube feeding or any type of enteral nutrition. In another embodiment, the dietary formula is polymeric diet formula or liquid dietary formula. However, other known dietary formulas may also be used. Typically, a refractometer is used to measure the Brix values, but other known methods may also be used.

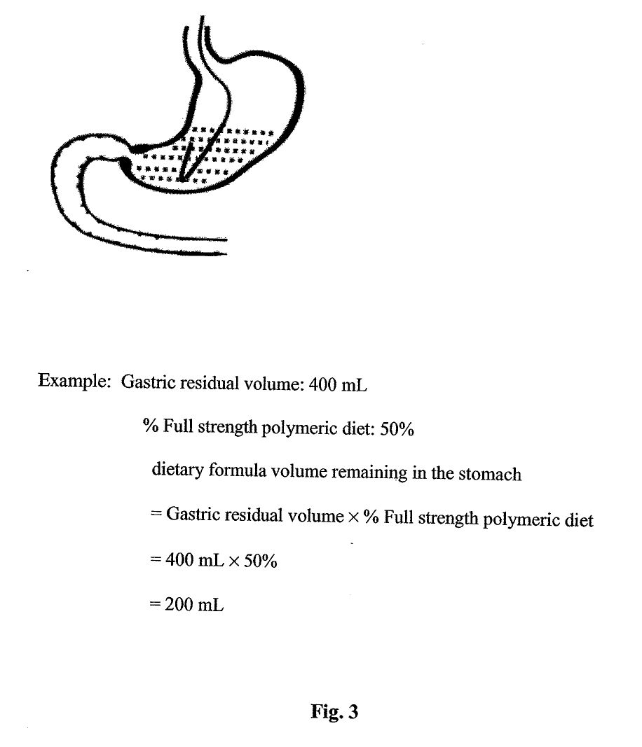

One embodiment of determining the amount of dietary formula remaining in the stomach is shown in FIG. 3. In this example, the gastric residual volume was measured or calculated at 400 ml. The dietary formula infused into the patient was 50% full strength polymeric diet formula. Thus, the volume of dietary formula remaining in the stomach at the point in time that the gastric residual volume was measured or calculated, is 200 ml.

A method of measuring gastric juice volume in stomach is also disclosed by measuring the dietary formula volume remaining in the stomach of a subject, calculating or measuring the gastric residual volume, and determining the value derived from the difference between the gastric residual volume and the dietary formula remaining in the stomach. The dietary formula volume remaining in the stomach can be obtained, for example, as disclosed above. However, other known methods of calculating dietary formula volume may also be used.

Based on the disclosed methods and the representative examples provided throughout, dietary ranges of nutrients (such as carbohydrate, protein, and fat) may be monitored by refractometry. The Brix value is a linear additive function of the concentration of nutrients present in a solution and Brix values correlate with the concentrations of dietary formula independent of pH, temperature, and the types of solutions. Thus, the Brix value can be used clinically and in research to monitor dietary formula concentrations, and therefore be used in clinical practice to evaluate dietary formula during storage, preparation, and administration.

The Brix value measurement of gastric contents can be used to monitor both gastric residual volume and food content in patients receiving enteral nutrition, e.g., by nasogastric feeding. The Brix value measurement of gastric juice can be used to monitor gastric emptying in patients receiving nasogastric feeding, thereby providing additional information to calculation of the aspirated gastric residual volume.

Because the Brix value measurements for various dilutions of the polymeric dietary have minimal variability in vitro, the disclosed methods permits bedside measurements with a high degree of reproducibility. In another application, saliva and gastric secretions have few dissolved substances and therefore have correspondingly low Brix value, close to that distilled water. Therefore, the disclosed methods can also be used to determine whether gastric contents are comprised predominately of dietary formula or digestive secretions in patients receiving polymeric dietary feeding.

Brix value measurement of gastric contents can be used in clinical practice, complementing the use and interpretation of gastric residual volumes, particularly for management of patients receiving nasogastric tube feeding. Gastric residual volume is determined by the dynamic balance between input (endogenous saliva plus gastric juice and exogenous formula) and output (gastric emptying) from the stomach. A low Brix value (especially if there is a steady decrease following bolus infusion) indicates a low concentration of formula remaining and implies adequate emptying of formula from the stomach. This may be confirmed using the dilution technique and the change in Brix value to determine calculated gastric residual volume and specific volume of formula remaining.

A method of monitoring gastric content emptying and tolerance in patients receiving dietary formula is further disclosed. The Brix value ratio, an alternative measure of the amount of dietary formula retained in the stomach, may be calculated by:

$$\frac{post \text{-} dilution Brix value}{pre \text{-} dilution Brix value}$$

The pre-dilution Brix value may be obtained from measuring the Brix value of a sample aspirated from the gastric contents of the stomach of a patient infused with dietary formula. After a known volume of water has been added to the gastric contents and reinstalled in the patient, the post-dilution Brix value may be obtained from reaspirating a sample and measuring the Brix value.

The specific component of gastric residual volume that is volume comprised of dietary formula may be determined using the equation:

$$\begin{matrix}Volume of dietary formula \\ remaining in stomach\end{matrix} = \frac{\begin{matrix}calculated gastric residual volume \times \\ \frac{( pre \text{-} dilution Brix values )}{0.24}\end{matrix}}{100}$$

### Alternative Method for Determining Gastric Residual Volume

An alternative method for determining gastric residual volumes is also described.

This method is particularly suitable for enabling clinicians to determine gastric residual volumes in patients whom have mainly gastric fluid in their stomachs or very low concentrations of dietary formula; although the previously described methods may alternatively be used.

Although not limited to use with such patients, this method is particularly applicable to the following patient groups:

1. Pre-surgery and Intensive Care Unit (ICU) patients. These patients may have a feeding tube, as in the case of typical ICU patients. A surgeon or anesthesiologists may order the aspiration of the entire stomach contents before surgery to determine the gastric residual volume. Such patients have generally fasted for at least 6 hours and, therefore, the concentration of soluble solids in the stomach is typically less than 3%.

2. Post surgery patients. This patient group may have had an “open” surgical procedure and the patient is then monitored for a build-up of gastric fluid. For example, a build-up of fluid increases the risk of aspirating the fluid with the associated adverse side effects.

3. Continuous enterally fed patients. These patients typically have low dietary formula concentrations within the stomach and the measurement of gastric residual volume may be ordered once per nursing shift and/or before each feeding cycle.

In each of the above exemplified patient groups, the gastric fluid may comprise, for example: dietary formula, gastric juices and/or saliva; and may have a concentration of soluble solids of less than approximately 5% at the time of measuring the gastric residual volume.

In accordance with this method (or process) of measuring gastric residual volume, a change in concentration of soluble solids in the stomach of a subject is caused by injecting a known concentration of a liquid composition composed of a water soluble solid into the stomach of a subject (e.g. a patient).

The concentration of soluble solids in the stomach, for example, the Brix value of the stomach or gastric contents, is measured to obtain a pre-dilution concentration (C1). A first sample as a portion of the gastric contents is removed from the stomach using any appropriate means; and the soluble solids concentration (or Brix value) of the portion may be measured using any appropriate means, such as by refractometry, a combination of refractometry and reflectometry, etc. Conveniently, the portion of gastric contents can be removed by way of a feeding tube, such as a nasogastric feeding tube.

Any size portion of the gastric contents may be removed as a pre-dilution sample in order to measure the concentration of soluble solids of the gastric contents. However, the method advantageously requires only a small pre-dilution sample, which may be a relatively small portion of the total gastric contents. Thus, the first sample may be less than 30 ml, suitably less than 20 ml, more suitably less than 10 ml, and still more suitably less than 5 ml. In some embodiments, the first sample is between 1 and 3 ml, for example, approximately 2 ml. Accordingly, the first sample may be less than approximately 50%, less than approximately 25%, less than approximately 10%, less than approximately 5%, or less than approximately 2% of the total gastric contents. It will be appreciated that the exact size of the sample and, hence, the proportion of the gastric contents removed from the stomach is not critical to the working of the invention. However, the invention benefits from the fact that, unlike conventional measurement techniques, it is not necessary to attempt to remove the entire gastric contents. The concentration of soluble solids (e.g. the Brix value) may, of course, be obtained from an in vitro sample (such as one previously obtained from a subject), although the sample is beneficially obtained directly from the stomach of the subject. Optionally, the first sample of gastric contents may be returned to the stomach of the subject.

A change in concentration of the gastric contents is caused by adding to the stomach of the subject (e.g. a patient) a “marker solution” of a known amount of a soluble solid in a known volume of liquid (i.e. solvent), using any suitable method. In some embodiments, the soluble solid is dissolved in water to a known concentration and injected into the stomach of the subject through a feeding tube. In other embodiments, the soluble solid may be dissolved in another suitable liquid, such as a dietary formula, sucrose solution, gastric juice and the like, provided that the concentration of soluble solids in the marker solution is known. By way of non-limiting examples, suitable solids for dissolution to create a marker solution include, for example, carbohydrates such as sucrose, polyethylene glycols such as PEG 3350, sucralose, and wheat dextrin. However, any other suitable material could be used to raise the quantity of soluble solids in the stomach.

The volume of water (or other liquid) used as a carrier to transport the soluble solid is not critical to the method of the invention, although, advantageously, the volume of the marker solution added to the stomach of the subject is as small as possible. For instance, the volume of marker solution infused is typically less than approximately 50 ml, suitably less than approximately 40 ml, more suitably less than approximately 30 ml, and still more suitably less than approximately 20 ml. Thus, in some embodiments, approximately 20 ml or 30 ml of a marker solution may be infused into the stomach of a patient in order to provide a change in the concentration of gastric contents. However, in advantageous embodiments, a smaller volume of marker solution may be infused, such as approximately 10 ml or less, for example, approximately 5 ml.

It will be appreciated that the concentration of soluble solids material in the marker solution may also affect the volume of marker solution infused in order to provide a change in the concentration of soluble solids in the stomach of the subject. That is, a relatively higher concentration of soluble solids in the marker solution may advantageously allow a relatively smaller volume of marker solution to be infused. Thus, the concentration of soluble solids material in the marker solution is conveniently at least 10%, suitably at least 20%, more suitably at least 25% and in some advantageous embodiments, at least 30%. For example, a saturated sucrose marker solution may contain approximately 33% sucrose. Hence, a sucrose marker solution used in accordance with the invention may conveniently comprise at least approximately 20% sucrose, more suitably at least approximately 25% sucrose, and in some embodiments may comprise at least approximately 30% or approximately 33% sucrose. When the soluble solid is a polyethylene glycol (PEG) material (e.g. PEG3350), the concentration of PEG in the marker solution may suitably be approximately 25% or more, such as approximately 30%, or approximately 31.5%; although, as already noted, any suitable concentration may be used.

In all cases, it is necessary that the concentration of the soluble solid in the marker solution that is infused into the stomach is known, and that the total volume of marker solution delivered to the stomach is also known. It will also be appreciated that the soluble solids material and the liquid/solvent are selected to be non-toxic to the patient or other subject into whom the marker solution is infused.

Having infused a known volume of a marker solution of soluble solids into the stomach of a subject, for example, by way of a feeding tube, the feeding tube may be flushed with a liquid having of low concentration of soluble solids, to flush any remaining marker solution within the infusion tube into the stomach. Suitably, the liquid used to flush any remaining marker solution into the stomach has a negligible concentration of soluble solids, such as water. Any suitable volume of water or other liquid may be used for flushing of the feeding tube, for example, although the volume selected should be sufficient to flush the tube, and not so high as to substantially dilute the gastric contents. Therefore, the volume of water (or other liquid) used to flush the tube is conveniently no more than about 5 times the volume of the marker solution that has been infused, and may suitably be 30 ml or less, 20 ml or less, or 10 ml or less. Thus, if 10 ml of marker solution is infused into the stomach of the subject, and 20 ml of water is used to flush a feeding tube used to infuse the marker solution, for example, effectively a total of 30 ml of solution would have been infused into the stomach along with the soluble solids material. It may be convenient to consider that the stomach (gastric) contents may be considered to have been “diluted” by the addition of the marker solution and by the addition of water (or other liquid) for flushing, even though the concentration of soluble solids in the stomach has been increased.

The entire contents of the stomach may be mixed, such as by repeated aspiration and re-infusion of a portion of the gastric contents. For example, a portion of the stomach contents can be aspirated and re-instilled three times, for example, by way of a syringe (e.g. 60 ml syringe) attached to a feeding tube.

A second (post-dilution) sample as a portion of the now diluted gastric contents is removed (e.g. by aspiration)—conveniently in the same manner as for the first sample—and a second concentration of soluble solids in the diluted gastric contents (e.g. a Brix value) is measured. As for the first sample removed, the second sample may comprise any proportion of the total gastric contents. However, the method only requires that a volume sufficient for the measurement of soluble solids (e.g. Brix value) is collected. Thus, the second sample may be a relatively small portion of the total gastric contents. For example, the sample volume may be less than 30 ml, less than 20 ml, less than 10 ml, or less than 5 ml. In some embodiments, the second sample is between 1 and 3 ml, for example, approximately 2 ml. Again, it will be appreciated that the exact size of the sample and, hence, the proportion of the gastric contents removed from the stomach is not critical to the working of the invention and, therefore, the invention benefits from the fact that it is not necessary to remove the entire gastric contents. Optionally, in instances in which the subject initially has a small volume of gastric contents prior to the performance of the method of the invention and it is desirable to maintain the subject at approximately that level, a major part (but not all) of the gastric contents (i.e. approximately 50% to 90%), or at least an equivalent volume to the volume of the solution of soluble solids added by the method, may be removed as a sample.

In some embodiments, it is advantageous to remove (e.g. aspirate) the first and second samples at a set time interval. For example, the first (pre-dilution) sample may be obtained up to 5 minutes prior to the collection of the second (post-dilution) sample. In some cases, the first sample may be obtained approximately 10 minutes, approximately 15 minutes or approximately 30 minutes prior to the collection of the second sample.

Having removed a second sample of gastric contents, the concentration of soluble solids, or in some embodiments the Brix value, of the second sample (C2) is determined. Conveniently, the concentration of soluble solids (e.g. the Brix value) is calculated in the same manner as for the first sample, for example, using refractometry, a combination of refractometry and reflectometry etc.

Thus, in one embodiment, the method comprises the steps:

Step 1: Remove (e.g. aspirate) a first (pre-dilution) sample as a portion of gastric contents (e.g. 1 to 3 ml) from the stomach of a subject.

Step 2: Measure the concentration of soluble solids in the first sample to obtain a first (or pre-dilution) concentration (C1).

Step 3: Optionally, mix an amount of soluble solids material with a liquid to form a “marker solution” comprising a solution of a known concentration of soluble solids. For example, about 3.33 g of a soluble solid material, such as sucrose, may be dissolved in 10 ml of water to obtain a 33.3% solution of the material.

Step 4: Deliver a set amount of the marker solution into the stomach of the subject, for example, by injection through the feeding tube.

Step 5: Optionally, flush the marker solution through the feeding tube into the stomach of the subject using a small quantity of liquid, for example, 10 to 20 ml of water.

Step 6: Optionally, mix the diluted gastric contents.

Step 7: Remove (e.g. aspirate) a second (post-dilution) sample as a portion of the gastric contents (e.g. 1 to 3 ml) from the stomach of the subject.

Step 8: Measure the concentration of soluble solids in the second sample to obtain a second (or post-dilution) concentration (C2).

Step 9: Calculate the gastric residual volume (GRV), for example, using Equation A below.

It should be noted that steps 3, 5 and 6 can be optional. For example, the marker solution may be made up in advance, such that step 3 is either not part of the method, or step 3 is carried out prior to step 1 and/or step 2.

The post-dilution gastric residual volume (GRV; i.e. after addition of the marker solution and optional flush liquid) may then be determined based on the concentrations of soluble solids in the first and second samples (i.e. C1 and C2), and the concentration and volume of the marker solution infused into the stomach. Conveniently, the gastric residual volume (GRV) may be calculated by: (i) determining a first difference between the concentration of soluble solids in the marker solution (Cm) and the concentration of soluble solids in the first sample (C1); (ii) determining a second difference between the concentration of soluble solids in the second sample (C2) and the concentration of soluble solids in the first sample; (iii) dividing the first difference by the second difference, and multiplying the quotient by the volume of the marker solution (Vm) infused into the stomach. It should be noted that the volume of marker solution (Vm) includes the volume of any further liquid that may have been used, for example, to flush the marker solution through the feeding tube into the stomach in optional step 5, above.

In a suitable method, the post-dilution gastric residual volume (GRV) can be calculated using Equation A, as follows:

GRV=Vm×[(Cm−C1)/(C2−C1)]

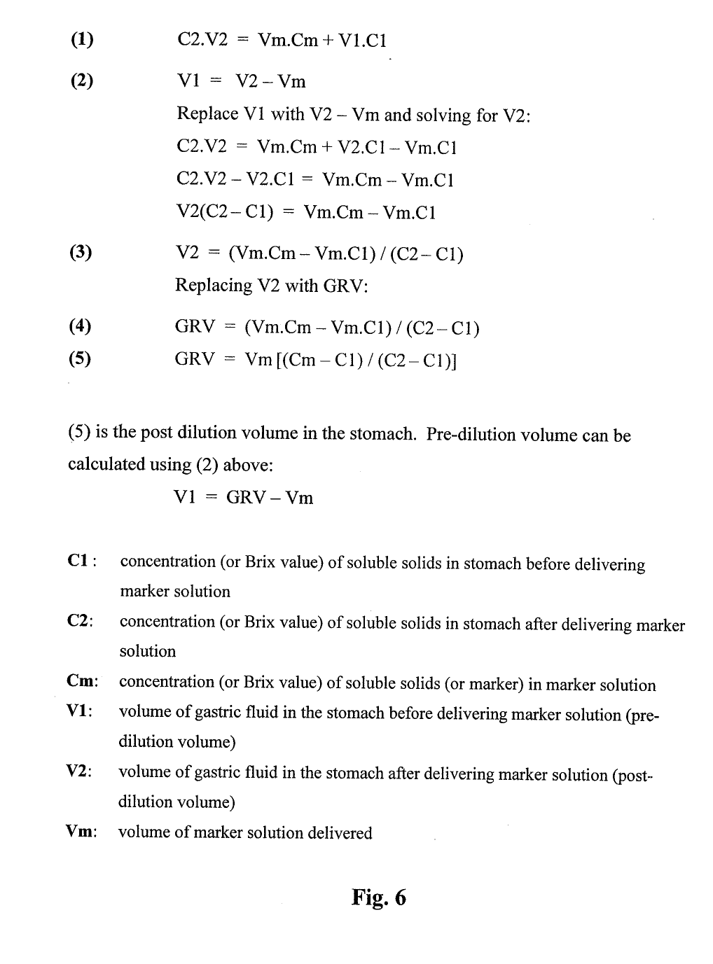

The derivation of Equation A is shown in FIG. 6. The concentrations, C1, C2 and Cm are measured in the same units (e.g. grams per litre) and are conveniently expressed as % soluble solids or in a Brix value (BV). Suitably, in one embodiment, the C1, C2 and Cm concentrations are expressed as % soluble solids. The gastric residual volume (GRV) and the volume of marker solution infused into the stomach are given in the same units, for example, in millilitres (ml).

In some cases, such as where the volume of the second sample is relatively large (e.g. more than 5 ml), it may be desirable to subtract the volume of the second sample that is removed from the stomach from the volume of the GRV calculated in Equation A.

Advantageously, this method allows the accurate determination of gastric residual volumes—especially in subjects (e.g. patients) whom have fasting or near-fasting levels of gastric contents. By “fasting” conditions it is meant that the subject has consumed no or substantially no food or drink orally into the stomach within the previous approximately 6 hours. Beneficially, a calculated gastric residual volume (GRV) determined in accordance with this method of the invention has an error of less than approximately 20%, suitably less than approximately 15%, more suitably less than approximately 10%, and still more suitably less than approximately 5% of the actual gastric residual volume (GRV).

Having determined the post-dilution gastric residual volume (GRV), for example, by the method of Equation A above, it is possible to determine the pre-dilution (i.e. the original) gastric residual volume (i.e. V1) using the equation:

V1=GRV−Vm

Since this method of the invention is equally applicable when the gastric contents contain dietary formula (such as in the case of ICU patients) and/or when the marker solution contains dietary formula, after determining the gastric residual volume (GRV) in the stomach of the subject, the volume of dietary formula (Vol. DF) in the stomach of the subject can then be calculated. For example, the volume of dietary formula in the stomach can be calculated using Equation B, as follows:

Vol. DF=GRV×(C1÷standard value for dietary formula)

In Equation B above, the concentration C1, is measured in the same units as for Equation A (e.g. expressed as % solids or Brix value (BV). The “standard value” for the dietary formula is the concentration of soluble solids or the Brix value (depending on which units were used in Equation A), in a concentrated (i.e. 100%) dietary formula. In one embodiment, wherein the polymeric dietary formula is Osmolite HN (Ross, Ohio, USA) the standard value is 24. Conveniently, gastric residual volume (GRV) can be expressed in millilitres (ml).

To assist in understanding the present application, the following examples are included and describe the results of a series of experiments. The following examples relating to this application should not be construed to specifically limit the application or such variations of the application, now known or later developed, which fall within the scope of the application as described and claimed herein.

### EXAMPLES

### Example 1

### Use of Brix value to Monitor Dietary Formula Concentration

### Materials and Methods

Brix values for nutrients such as minerals, vitamins mixtures, carbohydrate, protein, fat, and polymeric dietary were determined with a refractometer (N.O.W 507-1, Nippon Optical Works; Tokyo, Japan). A solution of minerals (Ringer's solution) was obtained from YF Chemical Corporation (Taipei, Taiwan), and consisted of sodium chloride (8.6 mg/ml), potassium chloride (0.3 mg/ml), and calcium chloride (0.33 mg/ml). Vitamins (Lyo-povigen, a parenteral vitamin mixture) was also obtained from YF Chemical Corporation (Taiwan), and contained vitamin A palmitate (12 IU/ml), vitamin D2 (1 IU/ml), vitamin E (0.005 IU/ml), vitamin C (0.5 mg/ml), vitamin B1 (0.05 mg/ml), vitamin B2 (0.01 mg/ml), vitamin B6 (0.015 mg/ml), niacinamide (0.1 mg/ml), and d-panthenol (0.025 mg/ml). Carbohydrate (Carb-aid, Corn starch) and protein (Whey-aid, lactoalbumin) were purchased from Nutritec-Enjoy Nutrition Center, Taiwan. Fat (Intralipid) was purchased from Frenius Kabi AB, Uppsala, Sweden. Full strength polymeric dietary (Osmolite HN, Ross, Ohio, USA) contained carbohydrate (17 g/100 ml), protein (5.3 g/100 ml), and fat (4.1 g/100 ml).

The Brix values were measured using a hand-held refractometer, whose Brix scale (% Brix) of 0-32 could be read in 0.2 increments. To measure the solute concentration, one or two drops of the specimen fluid were placed in a designated window. The refractometer was calibrated with distilled water before each measurement.

### Statistical Analysis

Results are presented as the mean ±SEM. Correlation coefficients following linear regression analysis were used to evaluate the relationship between Brix values and dietary formula concentrations. Differences were considered statistically significant when P<0.05.

### Results

### Brix Values of Nutrients

The Brix values of nutrients and dietary formula are listed in Table 1. Distilled water, minerals, and vitamins contained little dissolved material and had correspondingly low Brix values of 0±0, 1.2±0.1, and 0.4±0.1, respectively. Carbohydrate (17 g/100 ml), protein (5.3 g/100 ml), fat (4.1 g#100 ml), and full-strength polymeric dietary had high concentrations of dissolved nutrients and correspondingly high Brix values of 12.1±0.6, 6.5±0.1, 6.0±0.1, and 23.5±0.1, respectively.

TABLE 1
Brix values of nutrients

| Nutrient | Brix value (% Brix) |
|---|---|
| Distilled water | 0 ± 0 |
| Minerals (Ringer's solution) | 1.2 ± 0.1 |
| Vitamins (parenteral vitamin mixture) | 0.4 ± 0.1 |
| Carbohydrate (17 g/100 ml) | 12.1 ± 0.6 |
| Protein (5.3 g/100 ml) | 6.5 ± 0.1 |
| Fat (4.1 g/100 ml) | 6.0 ± 0.1 |
| Polymeric diet (full-strength) | 23.5 ± 0.1 |

Results presented are mean ± SD. Polymeric diet (Osmolite HN).

Molar Refractivities in Solution of Mixed Nutrients Were Additive. Table 2 shows Brix values of pure nutrients (such as carbohydrate, protein, and fat) and mixtures of these. Three dilutions (50%, 100% and 200% of the starting concentration) of carbohydrate, protein, and fat were made with distilled water. The Brix value was a linear additive function of the solute concentration, regardless of whether the solute was carbohydrate, protein, or fat.

TABLE 2
Brix values of pure and mixed nutrients

| Nutrient | Brix value (% Brix) |
|---|---|
| Carbohydrate (8.5 g/100 ml) | 6.8 ± 0.6 |
| Carbohydrate (17 g/100 ml) | 12.4 ± 0.6 |
| Carbohydrate (34 g/100 ml) | 25.8 ± 0.6 |
| Protein (2.6 g/100 ml) | 3.1 ± 0.1 |
| Protein (5.3 g/100 ml) | 6.3 ± 0.1 |
| Protein (10.5 g/100 ml) | 11.4 ± 0.1 |
| Fat (2.1 g/100 ml) | 3.1 ± 0.1 |
| Fat (4.1 g/100 ml) | 6.0 ± 0.1 |
| Fat (8.2 g/100 ml) | 11.9 ± 0.1 |
| Carbohydrate^{a} + protein^{b} | 19.2 ± 0.1 |
| Carbohydrate^{a} + protein^{b} + fat^{c} | 23.0 ± 0.1 |

^{a}Carbohydrate: 17 g/100 ml.
^{b}Protein: 5.3 g/100 ml.
^{c}Fat: 4.1 g/100 ml.
Results presented are mean ± SD.

For nutrient solutions, consisting of some combination of carbohydrate (17 g/100 ml), protein (5.3 g/100 ml), and fat (4.1 g/100 ml), Brix value was also an additive function of the component concentration. For example, a mixture of carbohydrate (17 g/100 ml) and protein (5.3 g/100 ml) had a Brix value of 19.2±0.1, and a mixture of carbohydrate (17 g/100 ml), protein (5.3 g/100 ml), and fat (4.1 g/100 ml) had a Brix value of 23.0±0.1, which was close to the Brix value (23.5±0.1) of full-strength polymeric diet.

Brix values of nutrients at various pHs and temperatures. Table 3 shows the effect of pH on the Brix value of serially diluted polymeric dietary formula Brix values correlated with the polymeric dietary formula concentration at each pH (P<0.001). However, the Brix values of the polymeric dietary formula were lower at pH 1 and 4 than at pH 7 and 8. The decrease of Brix value may be due to the protein denaturation that occurs in very acid solutions. Table 4 shows the effect of temperature on the Brix values of serially diluted polymeric dietary formula. The Brix values correlated with the concentrations of dietary formula at all temperatures investigated (P<0.001).

TABLE 3
Brix values of polymeric diet at various pH values

| | % Full-Strength of polymeric diet | | | | | | |
|---|---|---|---|---|---|---|---|
| pH | 0 | 12.5 | 25 | 50 | 75 | 100 | R² |
| pH 1 | 0 ± 0 | 3.2 ± 0.3 | 5.0 ± 0.1 | 9.6 ± 0.8 | 13.1 ± 0.3 | 17.2 ± 0.1 | 0.98 |
| pH 4 | 0 ± 0 | 2.1 ± 0.1 | 4.2 ± 0.2 | 9.0 ± 1.0 | 12.4 ± 0.3 | 16.8 ± 0.1 | 0.98 |
| pH 7 | 0 ± 0 | 3.0 ± 0.1 | 6.1 ± 0.1 | 12.3 ± 0.1 | 17.9 ± 0.1 | 23.5 ± 0.1 | 1.00 |
| pH 8 | 0 ± 0 | 3.0 ± 0.1 | 6.1 ± 0.1 | 12.3 ± 0.1 | 17.8 ± 0.2 | 23.4 ± 0.1 | 0.99 |

Brix values (% Brix) of polymeric diet (Osmolite HN) diluted in distilled water at adjusted to different pH values. Results are presented as means ± SEM.

TABLE 4
Brix values of polymeric diet at various temperatures

| | % Full-strength of polymeric diet | | | | | | |
|---|---|---|---|---|---|---|---|
| Temp. | 0 | 12.5 | 25 | 50 | 75 | 100 | R² |
| 4° C. | 0 ± 0 | 3.2 ± 0.1 | 6.1 ± 0.2 | 12.0 ± 0.1 | 17.8 ± 0.4 | 23.4 ± 0.1 | 0.99 |
| 25° C. | 0 ± 0 | 3.0 ± 0.1 | 6.0 ± 0.1 | 12.0 ± 0.2 | 17.8 ± 0.2 | 23.2 ± 0.1 | 0.99 |
| 37° C. | 0 ± 0 | 3.0 ± 0.1 | 6.1 ± 0.1 | 12.1 ± 0.1 | 18.3 ± 0.1 | 23.5 ± 0.3 | 0.99 |

Brix values (% Brix) of polymeric diet (Osmolite HN) diluted in distilled water equilibrated to different temperatures. Results are presented as means ± SEM.

Brix values of polymeric dietary in fasting gastric juice. Table 5 shows the effect of gastric juice dilution on the Brix value of the polymeric dietary formula. The Brix value measurements of polymeric dietary in fasting gastric juice were made at 5, 30, 120 and 240 min, respectively. Brix values correlated with the concentration of polymeric dietary diluted in fasting gastric juice at each time with minimal variability (R²<0.98). Therefore, polymeric dietary formula concentration in gastric contents can be estimated on the basis of the linear regression equation: Full strength polymeric diet % concentration=Brix value÷0.24, wherein 0.24 is the slope of serially diluted dietary formula concentration. For example, a Brix value of 6.0 and 12 in gastric contents corresponds to a 25% and 50% full-strength polymeric dietary concentration, expressed as % concentration of dietary formula.

TABLE 5
Table 5 Brix values of polymeric diet in gastric juice.

| | % Full-strength polymeric diet | | | | | | |
|---|---|---|---|---|---|---|---|
| Time (min) | 0 | 12.5 | 25 | 50 | 75 | 100 | R² |
| 5 | 2.0 ± 0.4 | 4.5 ± 0.3 | 6.8 ± 0.4 | 12.3 ± 0.8 | 18.0 ± 0.2 | 23.2 ± 0.4 | 0.99 |
| 30 | 2.1 ± 0.4 | 4.6 ± 0.4 | 6.8 ± 0.5 | 12.2 ± 0.7 | 18.0 ± 0.2 | 23.2 ± 0.1 | 0.98 |
| 120 | 2.0 ± 0.4 | 4.7 ± 0.3 | 6.9 ± 0.6 | 12.0 ± 0.7 | 17.8 ± 0.5 | 23.2 ± 0.1 | 0.98 |
| 240 | 2.0 ± 0.4 | 4.8 ± 0.5 | 7.0 ± 0.6 | 12.0 ± 0.8 | 17.6 ± 0.3 | 23.2 ± 0.1 | 0.98 |

Brix values (% Brix) of polymeric diet (Osmolite HN) diluted in fasting gastric juice.
Results are presented as means ± SEM (n = 6).

### Example 2

### Monitoring Bolus Nasogastric Feeding by the Brix Value Determination of Gastric Contents and Residual Volume Measurement of Gastric Contents

### Materials and Methods

All BV measurements were made using a hand-held refractometer (Model N.O.W. 507-1, Nippon Optical Works, Tokyo, Japan), whose Brix scale of 0-32 could be read in 0.2 increments. The refractometer was calibrated with distilled water before each measurement. One or two drops of the specimen fluid were placed on a designated window for observation, all measurements made at room temperature using natural light. In this way, the concentration of soluble solids in solution was measured at the bedside for each specimen.

Brix values for a polymeric (Osmolite HN, Abbott Laboratories, Columbus, Ohio) and five solutions (distilled water, 0.9% sodium chloride, 5% dextrose, fasting saliva, and gastric juice) were determined with the refractometer. Each liquid was evaluated six times. Serial dilutions of the polymeric formula (100%, 50%, 25%, 12.5%, 6.2%, and 0%) were made with three of the solutions (distilled water, saliva, and gastric juice) and is shown in the table below. The Brix value was measured in vitro, again performing six separate evaluations for each dilution of the polymeric formula.

The Brix Values for the polymeric diet diluted with different solutions.

| | % Full strength of polymeric diet (Osmolite HN) | | | | | |
|---|---|---|---|---|---|---|
| Solution | 0 | 6.2 | 12.5 | 25 | 50 | 100 |
| Distilled water | 0 ± 0 | 2.1 ± 0.4 | 3.5 ± 0.8 | 6.0 ± 0.9 | 12.2 ± 0.8 | 23.2 ± 0.3 |
| Saliva | 1.3 ± 0.4 | 2.2 ± 0.6 | 4.4 ± 0.6 | 7.3 ± 0.7 | 12.4 ± 0.9 | 23.2 ± 0.5 |
| Gastric juice | 1.9 ± 0.6 | 4.4 ± 1.4 | 5.4 ± 1.3 | 7.8 ± 1.4 | 13.3 ± 1.2 | 23.2 ± 0.4 |

Results are presented as, mean values for six separate determinations ± SEM.

Patients receiving bolus nasogastric feeding were used in this study. All subjects were fed the fill strength polymeric dietary via a 14 French nasogastric feeding tube. The polymeric dietary composition was 16.7% protein, 54.3% carbohydrate, and 29.0% lipid. Caloric requirements were calculated using the Harris-Benedict equation. A total of 250 ml of the polymeric dietary was administered by bolus infusion every 3-6 hours in the 24 hours prior to the study. Aspirated gastric residual volumes were obtained before each bolus feed by aspiration of the feeding tube. Aspirated gastric residual volumes were obtained first in the supine position, and then in the right lateral decubitus position. Following this preliminary period of monitoring, patients were arbitrarily divided into two groups based on conventional use of aspirated gastric residual volume; patients with low gastric residual volumes (<75 ml) were placed in group 1, and patients with higher gastric residual volumes (>75 ml on at least two occasions) were placed in group 2. In total, there were 25 subjects in group 1 (age: range 59-84, mean ±SD=75.9±6.6 years) and 18 patients in group 2 (age: range=44-79, mean ±SD=70.6±10.6 years).

After overnight fasting, all remaining gastric juice was aspirated from the stomach via the nasogastric tube using a 60 ml syringe. Then, all subjects received bolus infusion of 250 ml of the polymeric diet. Immediately after feeding, an attempt was made to thoroughly mix the food content in the stomach by aspirating and reinfusing of the nasogastric tube three times with a 60 ml syringe.

Sequential Brix value determinations were made on 2 ml samples of gastric contents at 0, 30, 60, 120, and 180 minutes intervals. At 180 minutes, any fluid remaining in the stomach was aspirated. Its volume was recorded as the aspirate gastric residual volume (Asp GRV), the Brix value measurement made, (pre-diluted BV) and then the contents were reinstilled into the stomach. An added dilutional 30 ml volume of distilled water was infused via the nasogastric tube. The stomach contents again were thoroughly mixed, and a 2 ml sample was obtained for a final BV measurement (Post Dilute BV). The Calculated GRV was determined using the equation: Calculated GRV×PreDilute BV (Calculated GRV+30 ml)×PostDilute BV. Specific volume of formula remaining at 180 minutes was defined by 2 equations; % Concentration=BV₁₈₀ₘᵢₙ/0.24 and Volume_{formula}=% Concentration×Calculated GRV.

The calculated GRV was determined by the equation:

$$\frac{Calc . GRV = 30 mi . Pre \text{-} Diluted BV}{Pre \text{-} Diluted B \text{-} Post \text{-} Diluted BV}$$

### Results

Results are presented as mean values±SEM. The Student's t test was used to assess differences in results between patients in group 1 and group 2. A p value of less than 0.05 was considered to be statistically significant.

Immediately following the 250 mL polymeric dietary bolus feeding, the mean Brix values, (BVs), of the gastric contents in vivo were shown to be lower both in group 1 and group 2 (19.6±1.0 versus 20.3±1.1, respectively) than the mean values for the polymeric formula made in vitro (23.2±0.3). This decrease in the Brix value most likely was due to the dilutional effect of endogenous gastric juices present in the stomach at the time of feeding.

The serial changes in Brix values for gastric content for the two groups following the bolus polymeric dietary feeding are shown in Tables 6 and 7. Mean serial Brix value measurements decreased in both groups after bolus feeding. For patients in group 2, the decrease was less, such that at 180 minutes patients in group 2 had a significantly higher mean Brix value for gastric contents than those patients in group 1 (10.1±0.7 versus 5.1±0.9, respectively, p<0.01). Tables 6 and 7 also show that the aspirated gastric residual volume at 180 minutes was significantly higher for patients in group 2 than for those in group 1 (72±12 versus 18±5 mL, respectively, p<0.01).

TABLE 6
Serial Brix values and gastric residual volumes (GRVs) for patients in group 1 (low GRVs)

| | Brix value of gastric juice | | | | | Residual volume (ml) | | |
|---|---|---|---|---|---|---|---|---|
| Patient | | | | | 180 | Aspirated | Calculated | Volume |
| no. | 0 min | 30 min | 60 min | 120 min | min | GRV | GRV | Formula |
| 1 | 13.9 | 12.2 | 5.3 | 8.5 | 9.6 | 30 | 23 | 9 |
| 2 | 22.3 | 14.3 | 14.3 | 14.3 | 14.8 | 0 | 3 | 2 |
| 3 | 23.4 | 19.5 | 13.7 | 10.3 | 11.6 | 70 | 115 | 56 |
| 4 | 22.6 | 14.6 | 13.2 | 12.4 | 10.1 | 10 | 31 | 13 |
| 5 | 11.9 | 10.4 | 8.2 | 4.6 | 4.9 | 10 | 215 | 44 |
| 6 | 21.5 | 13.5 | 12.3 | 9.4 | 8.5 | 30 | — | — |
| 7 | 22.4 | 13.2 | 12.5 | 7.2 | 8.7 | 20 | — | — |
| 8 | 23.3 | 20.4 | 14.3 | 10.3 | 9.1 | 79 | 68 | 26 |
| 9 | 14.5 | 11.3 | 9.4 | 5.4 | 5.3 | 32 | 92 | 20 |
| 10 | 12.9 | 10.6 | 8.3 | 5.3 | 4.3 | 12 | 185 | 33 |
| 11 | 13.3 | 9.2 | 8.1 | 5.0 | 4.2 | 10 | — | — |
| 12 | 14.8 | 10.3 | 8.3 | 6.4 | 4.8 | 15 | — | — |
| 13 | 17.6 | 12.3 | 10.4 | 7.4 | 4.6 | 70 | 62 | 12 |
| 14 | 17.7 | 12.4 | 9. | 7.5 | 4.4 | 78 | 80 | 15 |
| 15 | 14.8 | 10.3 | 8.3 | 6.4 | 4.8 | 15 | — | — |
| 16 | 20.2 | 18.4 | 15.3 | 4.6 | 3.5 | 8 | 45 | 7 |
| 17 | 17.2 | 14.5 | 10.2 | 4.4 | 2.6 | 32 | — | — |
| 18 | 16.5 | 12.5 | 9.3 | 3.6 | 2.5 | 30 | — | — |
| 19 | 16.4 | 12.4 | 9.3 | 3.5 | 2.3 | 5 | — | — |
| 20 | 22.3 | 14.4 | 10.0 | 5.4 | 2.9 | 9 | 15 | 2 |
| 21 | 22.3 | 21.2 | 20.5 | 3.3 | 2.7 | 1 | — | — |
| 22 | 20.9 | 17.4 | 14.3 | 3.7 | 2.3 | 5 | 47 | 4 |
| 23 | 26.2 | 16.3 | 14.1 | 12.3 | 2.0 | 8 | 30 | 3 |
| 24 | 23.8 | 12.7 | 10.6 | 4.3 | 1.5 | 0 | 15 | 1 |
| 25 | 26.0 | 15.6 | 15.2 | 13.4 | 1.3 | 0 | 48 | 3 |
| Mean ± SEM | 19.6 ± 1.0 | 14.0 ± 0.8 | 11.4 ± 0.8 | 7.1 ± 0.8 | 5.1 ± 0.9* | 18 ± 5* | 67 ± 15* | 6 ± 2* |

(*p < 0.05 for mean values group 1 versus group 2)

TABLE 7
Serial Brix values and gastric residual volumes (GRVs) for patients in group 2 (higher GRVs)

| | Brix value of gastric juice | | | | | Residual volume (ml) | | |
|---|---|---|---|---|---|---|---|---|
| Patient | | | | | 180 | Aspirated | Calculated | Volume |
| no. | 0 min | 30 min | 60 min | 120 min | min | GRV | GRV | Formula |
| 1 | 16.4 | 14.5 | 11.2 | 11.3 | 12.3 | 15 | 89 | 46 |
| 2 | 13.4 | 12.4 | 12.4 | 8.5 | 9.3 | 30 | 26 | 10 |
| 3 | 17.8 | 14.1 | 12.4 | 13.4 | 12.3 | 35 | 138 | 71 |
| 4 | 22.1 | 19.2 | 16.3 | 14.4 | 14.4 | 190 | 197 | 118 |
| 5 | 19.2 | 18.2 | 16.2 | 15.1 | 11.2 | 37 | 82 | 38 |
| 6 | 23.3 | 19.2 | 16.5 | 15.5 | 13.0 | 210 | 165 | 89 |
| 7 | 20.2 | 16.6 | 18.2 | 15.3 | 10.6 | 47 | 215 | 95 |
| 8 | 29.1 | 25.0 | 22.1 | 15.5 | 14.2 | 65 | 79 | 47 |
| 9 | 29.0 | 24.5 | 20.3 | 14.7 | 14.2 | 55 | 55 | 33 |
| 10 | 23.1 | 19.3 | 13.3 | 10.2 | 11.4 | 70 | 125 | 60 |
| 11 | 23.3 | 17.2 | 14.4 | 12.3 | 11.2 | 74 | 104 | 49 |
| 12 | 12.5 | 9.3 | 7.7 | 6.2 | 5.5 | 65 | 108 | 25 |
| 13 | 21.2 | 17.4 | 13.7 | 6.3 | 8.6 | 60 | 93 | 33 |
| 14 | 21.5 | 18.4 | 14.6 | 10.2 | 8.2 | 70 | 68 | 23 |
| 15 | 14.2 | 12.5 | 8.8 | 6.7 | 5.1 | 75 | — | — |
| 16 | 22.2 | 18.5 | 12.2 | 8.6 | 5.3 | 85 | — | — |
| 17 | 15.2 | 14.2 | 12.2 | 8.3 | 9.1 | 40 | 48 | 18 |
| 18 | 22.4 | 17.2 | 10.2 | 8.8 | 6.7 | 65 | — | — |
| Mean ± SEM | 20.3 ± 1.1 | 17.3 ± 1.0 | 14.0 ± 0.9 | 11.3 ± 0.8 | 10.1 ± 0.7* | 72 ± 12* | 106 ± 14* | 50 ± 8* |

(*p < 0.05 for mean values group 2 versus group 1)

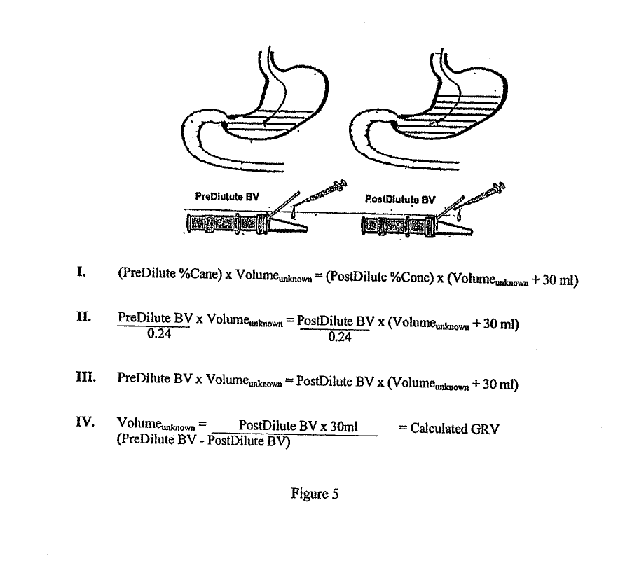

However, conventional use of GRV obtained by aspiration via a syringe may be inaccurate and unreliable in measuring true volume of contents present in the stomach at any given time. The dilution technique (determining BVs before and after addition of a known volume of water, e.g., 30 ml of distilled water) takes advantage of the relationship between the % Concentration of formula at any dilution and the measured BVs shown in FIG. 2, and may be used to calculate the true volume of contents and the specific volume of formula remaining in the stomach. As shown in FIG. 5, it is empiric that while an absolute amount of formula in the stomach does not change with the added dilutional 30 ml volume of distilled water, the total volume of gastric contents increases while the % concentration of formula and the corresponding BV decreases. The original volume of gastric contents present in the stomach before dilution (volume unknown) was derived from the change in the BV following dilution (FIG. 5). The amount of formula remains constant through dilution and is described by equation (1) in FIG. 5. Substituting the product BV/0.24 for % Concentration generates equation (2), both sides of which may be multiplied by 0.24 to derive equation (3). Solving for the volume unknown volume produces equation (4) and provides a value corresponding to the Calculated GRV.

The mean calculated gastric residual volume was shown to be significantly higher for patients in group 2 than for those in group 1 (106±14 versus 67±15 mL, respectively, p<0.05) (Tables 6 and 7). Using the final pre-diluted BV (i.e., the Brix value before water dilution) to derive the % concentration of the dietary formula, in combination with the calculated gastric residual volume (Calc. GRV), the specific volume of dietary formula of dietary present at 180 minutes was determined for both groups. The volume of dietary formula remaining was significantly higher for patients in group 2 compared to those in group 1 (50±8 versus 6±2 mL, respectively, p<0.05) (Tables 6 and 7).

As shown in Tables 6 and 7, use of refractometry in combination with conventional calculation of gastric residual volume identified 4% (1125) of patients in group 1 with low gastric residual volumes who might have possible gastric dysmotility (>20% of the initial 250 mL bolus volume of formula remaining at 180 minutes). Use of refractometry together with conventional measurement of gastric residual volumes indicated that 72% (13/18) of patients in group 2 with higher gastric residual volumes had sufficient gastric emptying (<20% of initial 250 mL volume of formula remaining).

The full strength dietary formula is rich in dissolved nutrients and displays a high BV of 23.2±0.3. By contrast, saliva and gastric secretions have few dissolved substances and therefore have correspondingly low BVs, close to that of 0.9% sodium chloride (Table 1). By evaluating the BV value, one may determine whether gastric contents are comprised predominately of dietary formula or digestive secretions in patients receiving dietary formula.

Monitoring GRV and Dietary Formula Concentration in Determining Feeding Tolerance

Brix value measurements and monitoring of GRVs and dietary formula concentrations are useful in evaluating feeding tolerance and gastric emptying. Embodying monitoring methods are exemplified in Table 8. A patient carrying a low GRV associated with low food retention (low Brix reading) would be interpreted to indicate that the formula is being emptied appropriately, and that there is no retention within the stomach. The risk for aspiration in the presence of low GRVs would be expected to be minimal, and the patient would be perceived as tolerating feeds. On the other hand, high GRV associated with high food retention would indicate the presence of delayed gastric emptying, an increased volume of gastric contents as a result of formula being retained in the stomach, and true feeding intolerance. However, patients carrying a low GRV but showing evidence of high food retention might be interpreted as having evidence of gastric dysmotility. In that case, the low GRV might represent a false negative screening monitor caused by the fact that the tip of the feeding tube is not in the pool of gastric juice, or the tip of feeding tube is adherent to the gastric mucosa. Low food retention by several BV determinations in a patient with high GRVs, may give some assurance that formula is being emptied effectively from the stomach. The high GRVs would thus represent a false positive screening monitor. Feeds could be continued as close clinical assessment is continued.

TABLE 8
Monitoring food content and residual volume simultaneously (prior to next feeding) in
patients receiving bolus nasogastric feeding.

| Residual | Formula | | |
|---|---|---|---|
| volume | Concentration | | Recommendation for clinical |
| (GRV) | (Brix Value) | Interpretation | practice |
| Low | Low | Good gastric emptying | Continue or even increase the |
| | | | tube feeding |
| High | High | Delayed gastric emptying | Stop or decrease the tube |
| | | | feeding, change from bolus-to |
| | | | continuous feeding, and/or |
| | | | switch to small bowel tube |
| | | | feeding |
| Low | High | May represent gastric | Check the true residual volume |
| | | dysmotility; aspirated | by water dilution technique, |
| | | GRV may be insensitive | close clinical monitoring to |
| | | to true GRV | assure tolerance |
| High | Low | May represent normal | Feeds could be continued with |
| | | emptying of formula; | close assessment of tolerance to |
| | | elevated aspirated GRV | enteral feeding; consider trial of |
| | | may reflect volume of | anti-secretory agents such as |
| | | endogenous secretions | proton pump inhibitor |

### Example 3

### Continuous Nasogastric Tube Feeding

### Monitoring by Brix Value and Conventional Gastric Residual Volumes

### Materials and Methods

After monitoring for 24 hours, 36 patients on continuous enteral tube feeding with a full strength (100%) polymeric dietary formula (Osmolite HN) were entered in this study and divided into 2 groups based on their pattern of conventional aspirated gastric residual volumes over the monitoring period. Patients with lower aspirated gastric residual volumes (<75 mL) were placed in Group 1, while patients with higher aspirated gastric residual volumes (>75 mL on at least 2 occasions) were placed in Group 2. Aspirated gastric residual volumes were obtained by aspiration of the feeding tube using a 60-ml syringe, first in the supine position, and then in the right lateral decubitus position. Upon entry, all gastric contents were aspirated, the volume recorded (aspirated gastric residual volume), Brix value measurements by refractometry performed, and the contents reinstilled but diluted with 30 mL additional water. Then a small amount was reaspirated, and repeat Brix value measurements were made. Three hours later, the entire procedure was repeated a second time.

The Brix values were measured using a hand-held refractometer, whose Brix scale of 0-32 could be read in 0.2 increments. The refractometer was calibrated with distilled water before each measurement. One or two drops of the specimen fluid were placed on a designated window for observation using daylight at room temperature. In this way, the concentration of soluble solids in solution was measured at the bedside for each specimen.

### Results

Results are presented as mean values ±SEM. The Student's t test was used to assess differences in results between patients in Group 1 and Group 2. A p value of less than 0.05 was considered to be statistically significant.

No patient in either group demonstrated nausea, vomiting, aspiration, or evidence of clear intolerance of enteral tube feeding. Table 9 shows the raw data of GRVs for the patients in Group 1. Patients in Group 1, with lower Asp GRVs based on the pre-study period of monitoring, continued to demonstrate very low Asp GRVs following entry into the study, with 93% (43/44) of the Asp GRVs obtained on the first and second measurements being <75 ml. In contrast, patients in Group 2 (again differentiated by higher GRVs on pre-study monitoring) continued to show higher Asp GRVs following entry into the study, with only 11% (3/28) of Asp GRVs on both measurements <75 ml. (Table 10). Mean aspirated GRV was significantly higher for those patients in Group 2 compared to those in Group 1 on both first (124±7 versus 14±2 ml, respectively, p<0.05) and second (75±10 versus 15±4 ml, respectively, p<0.05) measurements.

TABLE 9
GRVs for Group 1 patients (low residual volume) receiving continuous tube feeding

| | | First measurement (ml) | | | Second measurement (ml) | | |
|---|---|---|---|---|---|---|---|
| Patient | Infusion rate | | Cal | Formula | Asp | | Formula |
| no. | (ml/hr) | Asp GRV | GRV | remaining | GRV | Cal GRV | remaining |
| 1 | 75 | 45 | 78 | 39 | 6 | 35 | 33 |
| 2 | 30 | 40 | 40 | 12 | 15 | 56 | 24 |
| 3 | 75 | 30 | 55 | 24 | 3 | 79 | 72 |
| 4 | 75 | 25 | 74 | 17 | 85 | 90 | 14 |
| 5 | 65 | 20 | 35 | 24 | 4 | 38 | 35 |
| 6 | 20 | 15 | 75 | 6 | 20 | 60 | 9 |
| 7 | 45 | 12 | 33 | 32 | 20 | 59 | 35 |
| 8 | 60 | 10 | 32 | 28 | 5 | 35 | 32 |
| 9 | 75 | 10 | 34 | 32 | 5 | 37 | 31 |
| 10 | 50 | 10 | 31 | 23 | 4 | 37 | 28 |
| 11 | 30 | 10 | 37 | 32 | 35 | 66 | 23 |
| 12 | 55 | 10 | 37 | 35 | 12 | 35 | 32 |
| 13 | 30 | 10 | 31 | 26 | 5 | 35 | 29 |
| 14 | 45 | 8 | 32 | 30 | 3 | 32 | 30 |
| 15 | 40 | 8 | 34 | 31 | 20 | 97 | 23 |
| 16 | 30 | 7 | 31 | 29 | 5 | 32 | 30 |
| 17 | 70 | 7 | 40 | 40 | 11 | 49 | 36 |
| 18 | 70 | 7 | 36 | 36 | 32 | 69 | 32 |
| 19 | 70 | 7 | 40 | 40 | 11 | 49 | 36 |
| 20 | 70 | 6 | 33 | 31 | 5 | 33 | 30 |
| 21 | 60 | 6 | 33 | 31 | 10 | 33 | 31 |
| 22 | 30 | 10 | 34 | 31 | 3 | 39 | 34 |
| Mean ± SEM | 53 ± 4 | 14 ± 2* | 41 ± 3* | 29 ± 2* | 15 ± 4* | 50 ± 4* | 31 ± 2 |

Legend:
GRV = gastric residual volume,
Asp GRV = aspirated gastric residual volume,
Cal GRV = calculated gastric residual volume
(*p < 0.05 for mean value Group 1 versus Group 2).

TABLE 10
GRVs for Group 2 patients (high-residual volume) receiving continuous tube feeding

| | | First measurement (ml) | | | Second measurement (ml) | | |
|---|---|---|---|---|---|---|---|
| Patient | Infusion rate | Asp | | Formula | Asp | | Formula |
| no. | (ml/hr) | GRV | Cal GRV | remaining | GRV | Cal GRV | remaining |
| 1 | 40 | 145 | 104 | 19 | 18 | 39 | 37 |
| 2 | 70 | 142 | 130 | 51 | 110 | 146 | 62 |
| 3 | 75 | 140 | 171 | 65 | 110 | 149 | 71 |
| 4 | 40 | 110 | 200 | 33 | 10 | 37 | 34 |
| 5 | 40 | 98 | 88 | 18 | 9 | 43 | 39 |
| 6 | 30 | 128 | 138 | 135 | 97 | 62 | 27 |
| 7 | 35 | 120 | 116 | 114 | 95 | 50 | 26 |
| 8 | 30 | 110 | 140 | 141 | 85 | 47 | 22 |
| 9 | 30 | 160 | 150 | 87 | 80 | 132 | 62 |
| 10 | 40 | 155 | 110 | 69 | 90 | 115 | 61 |
| 11 | 30 | 138 | 132 | 75 | 86 | 117 | 51 |
| 12 | 70 | 130 | 180 | 72 | 100 | 180 | 77 |
| 13 | 30 | 85 | 193 | 72 | 78 | 119 | 47 |
| 14 | 30 | 78 | 282 | 110 | 80 | 193 | 72 |
| Mean ± SEM | 42 ± 4 | 124 ± 7* | 152 ± 13* | 76 ± 10* | 75 ± 10* | 102 ± 15* | 49 ± 5* |

Legend:
GRV = gastric residual volume,
Asp GRV = aspirated gastric residual volume,
Cal GRV = calculated gastric residual volume
(*p < 0.05 for mean value Group 1 versus Group 2).

Tables 11 and 12 show the pattern of the first and second Brix value measurements pre- and post-dilution. In general, patients in Group I tended to show of pattern of high pre-dilution Brix values dropping further to lower post-dilution Brix values than those patients in Group 2 (which showed the opposite pattern low pre-dilution Brix values dropping to a less extent to higher post-dilution Brix values). Only the difference in post-dilution Brix values between the two groups on both measurements reached statistical significance.

This pattern in Group 1 patients suggested that gastric contents were comprised of enteral formula of fairly high concentration (as evidenced by pre-dilution Brix values close to the in vitro Brix values of 23.2 for full strength Osmolite HN) that was of very small volume (as evidenced by the tremendous drop in Brix values with dilution by a small 30 ml volume of distilled water). The opposite pattern in Group 2 patients, in contrast, suggested greater dilution by endogenous secretions (as evidenced by pre-dilution Brix values less than the Brix value of 23.2 for full strength Osmolite HIS and greater total volume (as evidenced by the lesser drop in Brix values with dilution by a small 30 ml volume of distilled water).

The mean Brix value ratios for the two groups reflected these distinct patterns. The mean Brix value ratio was significantly higher for those patients in Group 2 compared to those in Group 1 on both the first (79±2% versus 20 t 4%, respectively, p<0.05) and second (59±7% versus 32±5%, respectively, p<0.05) measurements. For those patients in Group 1, Brix value ratios on all measurements for all patients were <70% (Table 11). For those patients in Group 2, the Brix value ratios on the first measurement were >70% on all but one patient #5) (Table 12). When measured the second time, 6 of the 14 patients (patients #1, 4-8) showed that the Brix value ratios had fallen to <70%.

TABLE 11
Brix values for Group 1 patients (low residual volume) receiving continuous tube feeding

| | First measurement (ml) | | | Second measurement (ml) | | |
|---|---|---|---|---|---|---|
| Patient | Pre-dilution | Post-dilution | Brix value | Pre-dilution | Post-dilution | Brix value |
| no. | Brix value | Brix value | ratio | Brix value | Brix value | ratio |
| 1 | 12.0 | 7.4 | 62% | 23.0 | 3.0 | 13% |
| 2 | 7.4 | 1.8 | 24% | 10.4 | 4.8 | 46% |
| 3 | 10.6 | 4.8 | 45% | 22.0 | 13.6 | 62% |
| 4 | 5.4 | 3.2 | 59% | 3.6 | 2.4 | 67% |
| 5 | 16.0 | 2.4 | 15% | 22.6 | 4.6 | 20% |
| 6 | 2.0 | 1.2 | 60% | 3.6 | 1.8 | 50% |
| 7 | 23.4 | 2.0 | 9% | 14.2 | 7.0 | 49% |
| 8 | 21.0 | 1.6 | 8% | 22.2 | 3.0 | 14% |
| 9 | 22.6 | 2.4 | 11% | 20.2 | 3.6 | 18% |
| 10 | 18.0 | 0.4 | 2% | 18.0 | 3.4 | 19% |
| 11 | 21.0 | 4.0 | 19% | 8.4 | 4.6 | 55% |
| 12 | 22.6 | 4.2 | 19% | 22.0 | 3.0 | 14% |
| 13 | 20.0 | 0.6 | 3% | 20.0 | 3.0 | 15% |
| 14 | 22.8 | 1.4 | 6% | 22.6 | 1.4 | 6% |
| 15 | 22.0 | 2.4 | 11% | 5.8 | 4.0 | 69% |
| 16 | 22.2 | 1.0 | 5% | 22.6 | 1.2 | 5% |
| 17 | 23.8 | 6.0 | 25% | 17.6 | 6.8 | 39% |
| 18 | 23.6 | 4.0 | 17% | 11.0 | 6.2 | 56% |
| 19 | 23.8 | 6.0 | 25% | 19.6 | 6.8 | 39% |
| 20 | 22.4 | 2.0 | 9% | 22.0 | 1.8 | 8% |
| 21 | 23.0 | 2.0 | 9% | 22.2 | 2.2 | 10% |
| 22 | 22.0 | 2.6 | 12% | 20.8 | 4.8 | 23% |
| Mean ± SEM | 18.5 ± 1.4 | 2.9 ± 0.4* | 20 ± 4%* | 16.9 ± 1.4 | 4.2 ± 0.6* | 32 ± 5%* |

*p < 0.05 for mean value Group 1 versus Group 2

TABLE 12
Brix values for Group 2 patients (high residual volume) receiving continuous tube feeding

| | First measurement (ml) | | | Second measurement (ml) | | |
|---|---|---|---|---|---|---|
| Patient | Pre-dilution | Post-dilution | Brix value | Pre-dilution | Post-dilution | Brix value |
| no. | Brix value | Brix value | ratio | Brix value | Brix value | ratio |
| 1 | 4.5 | 3.2 | 71% | 22.7 | 5.4 | 24% |
| 2 | 9.5 | 7.3 | 77% | 10.2 | 8.1 | 79% |
| 3 | 9.1 | 7.5 | 82% | 11.4 | 9.1 | 80% |
| 4 | 4.0 | 3.4 | 85% | 22.3 | 4.0 | 18% |
| 5 | 5.0 | 3.3 | 66% | 22.0 | 6.5 | 30% |
| 6 | 23.5 | 18.4 | 78% | 10.4 | 5.4 | 52% |
| 7 | 23.6 | 17.5 | 74% | 12.4 | 5.0 | 40% |
| 8 | 24.2 | 19.0 | 79% | 11.0 | 4.0 | 36% |
| 9 | 14.0 | 11.2 | 80% | 11.4 | 8.8 | 77% |
| 10 | 15.0 | 10.9 | 73% | 12.7 | 9.4 | 74% |
| 11 | 13.6 | 10.5 | 77% | 10.5 | 7.8 | 74% |
| 12 | 9.6 | 8.0 | 83% | 10.2 | 8.5 | 83% |
| 13 | 9.0 | 7.6 | 84% | 9.5 | 7.1 | 75% |
| 14 | 9.4 | 8.4 | 89% | 9.0 | 7.6 | 84% |
| Mean ± SEM | 12.4 ± 1.9 | 9.7 ± 1.4* | 79 ± 2%* | 13.3 ± 1.3 | 6.9 ± 0.5* | 59 ± 7%* |

*p < 0.05 for mean value Group 2 versus Group 1

As a whole, patients in Group 1 had a volume of formula estimated to be remaining on both measurements that was very low, reinforcing the pattern shown by Brix value ratios. (Table 9). In one patient (patient #22) where the volume of formula was greater than the hourly feeding rate, aspirated GRV and calculated GRV were <40 ml. Overall, 95% (21/22) of the Group 1 patients had a volume of formula remaining on both measurements that was less than the hourly feeding rate.

In contrast to those patients in Group 1, patients in Group 2 showed evidence of reduced gastric emptying and greater volume of retained formula (Table 10). Comparing the formula estimated to be remaining with the hourly infusion rate, six patients (patients #9-14) showed concurrence on both measurements for evidence of retention of formula and decreased or impaired gastric emptying. For these six patients, the volume of formula remaining was estimated to be greater than the hourly infusion rate on both measurements. In these six, both the first and second Brix value ratios were >70%. Traditional Asp GRV, however, was insensitive and failed to identify these patients, being [100 ml on two of the six first round of measurements, and six out of six on the second round of measurements. Three patients from Group 2 (patients #6-8), who on initial measurement showed retention of formula, improved on follow-up measurement indicating adequate gastric emptying of the formula. Five patients could be identified (patients #1-5) with high Asp GRVs from Group 2 who had a volume of formula remaining estimated to be less than the hourly infusion rate, assuring adequate gastric emptying all along.

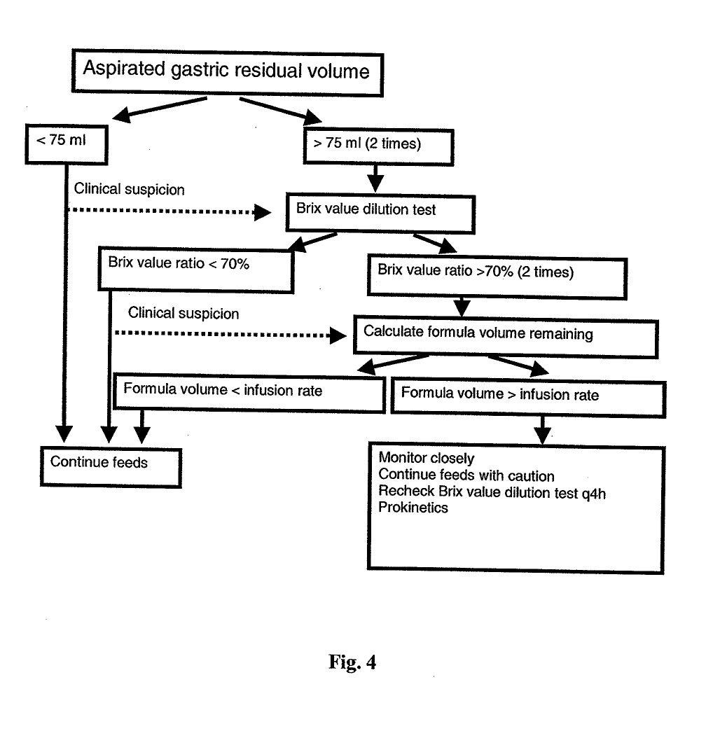

FIG. 4 and the results of this study demonstrate how refractometry may be used in conjunction with conventional measurement of aspirated gastric residual volumes to create a strategy by which to monitor patients on continuous gastric feeding. As shown in this study, patients who continue to have lower aspirated gastric residual volumes (for example, less than 75 mL) appear to be at low risk, and should have good tolerance and sufficient gastric emptying, and do not require refractometry assessment (unless clinical suspicion of intolerance increases, based on abdominal distension, reduced passage of gas and stool, increasing nausea, or regurgitation and vomiting). If patients are found to have higher aspirated gastric residual volumes (for example, greater than 75 mL on more than two occasions) then a Brix value dilution test is performed. Of course, the ordinary clinician or practitioner is skilled in determining whether the aspirated gastric residual volume is relatively higher or lower, dependent upon the clinical setting of the subject (patient).

As noted elsewhere in this application, a Brix value dilution test may be performed by measuring the Brix value of a gastric content sample to obtain a pre-dilution Brix value, adding a known volume of water to the gastric content sample to form a post-dilution sample, infusing the post-dilution sample into the stomach of a subject patient), aspirating the post-dilution sample from the stomach, measuring the Brix value of the aspirated post-dilution sample to obtain a post-dilution Brix value, and dividing the post-dilution Brix value by the pre-dilution Brix value to obtain a Brix value ratio.

In one embodiment, the Brix value ratio is less than 70%, as this study has shown, there is a low likelihood for retention of formula and feeds may continue. In another embodiment, if the Brix value ratio is greater than 70% on for example, more than 2 occasions (e.g., performed every 4 hours), the clinician calculates the volume of dietary formula remaining in the stomach. The number of times that the Brix value ratio is greater than 70% and the time interval between performing successive tests is dependent on the clinical setting of the patient and easily determined by the ordinary practitioner. The finding that the volume of dietary formula remaining in the stomach is greater than the hourly infusion rate alerts the clinician to the possibility of delayed gastric emptying with retention of formula, and that feeds may be continued with caution under close observation (for example, continuing to perform Brix value dilution tests with aspirated gastric residual volumes every 4 hours or some other determined time period).

### Example 4

### Determination of Gastric Residual Volume (GRV)

### Materials and Methods

Forty-three patients receiving bolus nasogastric feeding were monitored for 24 hours prior to entry into the study, and then divided into two groups based on conventional use of GRV; patients with low GRVs (<75 ml) were placed in group 1, while patients with higher GRVs (>75 ml) were placed in group 2. All subjects were given 250 ml of polymeric formula by bolus nasogastric infusion, followed by Brix value (BV) measurement of gastric contents at 0, 30, 60, 120, and 180 minutes. All gastric fluid was aspirated after 180 minutes of feeding; the volume was recorded (Aspirate GRV) and BV made (PreDilute BV), then reinstilled with an added 30 ml of dilutional water, after which a final aspiration and BV 0.5 measurement (PostDilute BV) was performed. Calculated GRV was determined by the equation: Calculated GRV×PostDilute BV=(Calculated GRV+30 ml)×PostDilute BV Specific volume of formula at 180 minutes was defined by 2 equations; % Concentration=BV₁₈₀ₘᵢₙ/0.24 and Volume_{formula}=% Concentration×Calculated GRV.

### Results

Serial BV measurements decreased in both groups after bolus feeding. For patients in group 2 the decrease was less, such that at 180 minutes the mean BV for gastric contents was, significantly higher than for those patients in group 1 (10.1 versus 5.1, respectively, p<0.01). Aspirate GRV, Calculated GRV, and Volume_{formula} present at 180 minutes was significantly greater for patients in group 2 compared to those in group 1. Use of refractometry in combination with traditional use of GRV identified 4% (1/25) of patients in group 1 with low GRVs who might have possible gastric dysmotility (>20% of initial 250 ml volume of formula remaining at 180 minutes), and assured that 72% (13/18) patients in group 2 with higher GRVs had sufficient gastric emptying (<20% of initial 250 ml volume of formula remaining).

### Example 5

### In Vitro determination of Gastric Residual Volume (GRV) Using Artificial Gastric Juice and Concentrated Sucrose Solution

This example shows, in an in vitro test experiment, how the method of the invention may be used to calculate gastric residual volumes (GRV) in a subject having gastric contents that contain a relatively low concentration of soluble solids by adding a solution having a relatively high concentration of soluble solids.

### Materials and Methods

Concentrations of soluble solids in test solutions of nutrients, such as artificial gastric juice and sucrose, were determined by Brix value measurements using a refractometer (ATAGO® Automatic Refractometer, SMART-1). The ATAGO® SMART-1 refractometer has a range of 0.0 to 95.0%; a Brix Value resolution of 0.1%; a Brix value accuracy of ±0.05%; a Brix value temperature compensation in the range of 41 to 104° F. (5 to 40° C.); and requires a minimum sample volume of only 0.1 ml. The refractometer was calibrated with distilled water before each measurement. One or two drops of the specimen fluid were placed on a designated window for observation, all measurements were made at room temperature (21° C.) in natural light.

A solution of artificial gastric juice was made by dissolving 2 g of NaCl, 3.2 g of Pepsin (1:3000 activity) and 7 ml of concentrated HCl in 100 ml of deionized water. The solution was then mixed and diluted to 1000 ml of deionized water, giving a concentration of approximately 1% soluble solids at a pH of approximately 1.2, to reflect the typical composition of gastric contents in a subject that has fasted (e.g. not orally consumed food or drink for approximately at least 6 hours). A marker solution, that is, a solution having a relatively high concentration of soluble solids was made by dissolving 7.44 g of sucrose in 30 ml water to form a 25% sucrose solution.

The concentration of soluble solids in the artificial gastric juice was measured using the ATAGO® SMART-1 device to obtain a pre-dilution concentration of soluble solids (C1). A series of “dilutions” were then made of 20 ml, 50 ml and 80 ml volumes of artificial gastric juice with 30 ml 25% sucrose (i.e. 7.44 g sucrose) and mixed thoroughly. A small sample (e.g. 1 ml) of the sucrose-artificial gastric juice mixture was then taken for measurement of the soluble solids in the post-dilution sample (C2), using the same refractometer. Finally, the total volume of solution in the sucrose-artificial gastric juice mixture (i.e. the hypothetical gastric residual volume) was calculated using Equation A.

### Results

The soluble solids concentrations of the artificial gastric juice and the sucrose marker solution are shown in Table 13. As indicated the artificial gastric juice had a relatively low concentration of soluble solids of 1.09%. In each case, the calculated gastric residual volume (GRV), based on the pre- and post-dilution soluble solids concentrations (C1 and C2), compare favourably with the actual volumes (“Total volume”), in all cases being within 10% of the actual volume. In one of the three tests samples, the % difference between the calculated and actual volumes was less than 5%.

TABLE 13
Calculated GRV based on artificial gastric juice and sucrose-based marker solution

| Artificial | | | | | Conc. | | | |
|---|---|---|---|---|---|---|---|---|
| gastric | Marker | | Conc. of | Conc. | of | | | |
| juice | solution | Total | marker | of first | second | | Volume | |
| volume, | volume, | volume, | solution, | sample, | sample, | Calculated | difference, | |
| V1 | Vm | V2 | Cm | C1 | C2 | GRV | GRV − V2 | % |
| (ml) | (ml) | (ml) | (%) | (%) | (%) | (ml) | (ml) | difference |
| 20 | 30 | 50 | 24.8 | 1.09 | 14.69 | 52.3 | 2.3 | 4.6% |
| 50 | 30 | 80 | 24.8 | 1.09 | 10.61 | 74.7 | −5.3 | −6.6% |
| 80 | 30 | 110 | 24.8 | 1.09 | 8.1 | 101.5 | −8.5 | −7.8% |

### Example 6

### In Vitro Determination of Gastric Residual Volume (GRV) Using Human Gastric Juice and Concentrated Sucrose Solution

This example demonstrates, in an in vitro test experiment, how the method of the invention may be used to calculate gastric residual volumes (GRV) in a subject having gastric contents that contain a relatively low concentration of soluble solids by adding a solution having a relatively high concentration of soluble solids.

### Materials and Methods

Concentrations of soluble solids in human fasted gastric juice and sucrose solution were determined by Brix value measurements using a refractometer (ATAGO® Automatic Refractometer, SMART-1), as described in Example 5.

Samples of human fasted gastric juice were obtained by aspiration from the stomach of 6 human subjects (samples 1 to 6) whom had fasted overnight (i.e. for at least 6 hours prior to collection of the sample). A sucrose marker solution was made by dissolving 10 g of sucrose in 30 ml water to form a 33.3% (approx) sucrose solution.

The concentration of soluble solids in the human fasted gastric juice samples was measured using the ATAGO® SMART-1 device to obtain pre-dilution concentrations of soluble solids (C1) for each sample. Then, each of the samples of human fasted gastric juice were mixed with 30 ml of the 33.3% sucrose solution (i.e. 10 g sucrose). A small sample (e.g. one or two drops) of each of the sucrose-human gastric juice mixtures was then taken for measurement of the soluble solids in the post-dilution samples (C2), using the same refractometer. As before, all measurements were taken at 21° C. Finally, the total volume of solution in the sucrose-human gastric juice mixture (i.e. the hypothetical gastric residual volume) was calculated using Equation A.

### Results

The results of this experiment are shown in Table 14. As indicated, the soluble solids concentrations in the human fasted gastric juice samples varied between 0.99 and 2.01%. As demonstrated in the “Volume difference” column, the maximum error in the calculated volume of the mixture in comparison to the actual volume of the mixture was 7.7 ml, which corresponded to an error on only 9.6% (see “% difference”). The average error over the remaining 5 tests was only 2.5 ml or 5.72% approximately. The results clearly show that the method of the invention can be used to accurately calculate the volume of a mixture of human gastric juice and sucrose solution, based on the measurements of soluble solids concentrations in relatively small samples as a portion of the solutions.

TABLE 14
Calculated GRV based on human gastric juice and sucrose-based marker solution

| | Artificial | | | | | Conc. | | | |
|---|---|---|---|---|---|---|---|---|---|
| | gastric | Marker | | Conc. of | Conc. | of | | | |
| | juice | solution | Total | marker | of first | second | | Volume | |
| | volume, | volume, | volume, | solution, | sample, | sample, | Calculated | difference, | |
| | V1 | Vm | V2 | Cm | C1 | C2 | GRV | GRV − V2 | % |
| | (ml) | (ml) | (ml) | (%) | (%) | (%) | (ml) | (ml) | difference |
| 1 | 7 | 30 | 37 | 33.3 | 1.31 | 28.10 | 35.8 | −1.2 | −3.2% |
| 2 | 8 | 30 | 38 | 33.3 | 0.99 | 27.86 | 36.1 | −1.9 | −5.1% |
| 3 | 9 | 30 | 39 | 33.3 | 1.26 | 27.28 | 36.9 | −2.1 | −5.3% |
| 4 | 20 | 30 | 50 | 33.3 | 1.40 | 21.68 | 47.2 | −2.8 | −5.6% |
| 5 | 50 | 30 | 80 | 33.3 | 1.35 | 14.61 | 72.3 | −7.7 | −9.6% |
| 6 | 18 | 30 | 48 | 33.3 | 2.01 | 23.60 | 43.5 | −4.5 | −9.4% |

### Example 7

### In Vitro determination of Gastric Residual Volume (GRV) Using Artificial Gastric Juice and PEG3350 Solution

This example demonstrates how the method of the invention may be used to calculate gastric residual volumes (GRV) in a subject having gastric contents that contain a relatively low concentration of soluble solids by adding a solution having a relatively high concentration of soluble solids.

### Materials and Methods

Concentrations of soluble solids in artificial gastric juice and PEG3350 solution were determined by Brix value measurements using a refractometer (ATAGO® Automatic Refractometer, SMART-1), as described in Example 5.

The artificial gastric juice solution was prepared as described in Example 5. The marker solution containing PEG3350 was made by dissolving 9.438 g (approx) of PEG3350 in 30 ml water, to form a 31.5% (approx.) solution.

The concentration of soluble solids in the artificial gastric juice was measured by placing one or two drops of solution on the reading window of the ATAGO® SMART-1 device to obtain pre-dilution concentrations of soluble solids (C1). Next, 30 ml of the 31.5% PEG3350 solution (9.438 g) was added and mixed thoroughly with each sample of the artificial gastric juice to obtain post-dilution mixtures. A small post-dilution sample (e.g. one or two drops) of each of the PEG3350-artificial gastric juice mixtures was then taken for measurement of the soluble solids in the post-dilution samples (C2), in the same way as the C1 sample. All measurements were taken at 21° C., and the total volume of solution in the sucrose-human gastric juice mixture (i.e. the hypothetical gastric residual volume) was calculated using Equation A.

### Results

The results of this experiment are shown in Table 15. The soluble solids concentration in the artificial gastric juice samples was 1.1%. It can be clearly seen that the “calculated” gastric residual volumes (GRV) in each test experiment were in good agreement with the actual volumes (see “Total volume”). The maximum difference between the calculated and actual volumes was 8.0 ml and the average percentage difference was approximately 5.95%. These results demonstrate that the method of the invention could be used to accurately calculate gastric residual volumes (GRV) of gastric contents using a marker solution comprising PEG3350, and significantly, only requiring small samples or portions of each of the solutions for measurements.

TABLE 15
Calculated GRV based on artificial gastric juice and PEG3350-based marker solution

| Artificial | | | | | Conc. | | | |
|---|---|---|---|---|---|---|---|---|
| gastric | Marker | | Conc. of | Conc. | of | | | |
| juice | solution | Total | marker | of first | second | | Volume | |
| volume, | volume, | volume, | solution, | sample, | sample, | Calculated | difference, | |
| V1 | Vm | V2 | Cm | C1 | C2 | GRV | GRV − V2 | % |
| (ml) | (ml) | (ml) | (%) | (%) | (%) | (ml) | (ml) | difference |
| 20 | 30 | 50 | 31.46 | 1.1 | 20.06 | 49.7 | −2.0 | −4.0% |
| 40 | 30 | 70 | 31.46 | 1.1 | 14.8 | 68.9 | −3.5 | −5.0% |
| 60 | 30 | 90 | 31.46 | 1.1 | 12.04 | 86.3 | −6.7 | −7.5% |
| 80 | 30 | 110 | 31.46 | 1.1 | 10.03 | 105.7 | −8.0 | −7.3% |

### Example 8

### In Vitro determination of Gastric Residual Volume (GRV) Using Artificial Gastric Juice Containing Dietary Formula and PEG3350 Solution

This example demonstrates how the method of the invention may be used to calculate gastric residual volumes (GRV) in a subject having gastric contents that contain a relatively low concentration of soluble solids but which also includes dietary formula, by adding a marker solution having a relatively high concentration of soluble solids.

### Materials and Methods

Concentrations of soluble solids in artificial gastric juice containing dietary formula and PEG3350 solution were determined by Brix value measurements using a refractometer (ATAGO® Automatic Refractometer, SMART-1), as described in Example 5. The artificial gastric juice solution was made by dissolving 2 g of NaCl, 3.2 g of Pepsin (1:3000 activity) and 7 ml of concentrated HCl in 100 ml deionized water. The solution was then mixed and diluted to 1000 ml of deionized water, giving a concentration of approximately 1% soluble solids at a pH of approximately 1.2. A 10% (v/v) solution of polymeric dietary formula (Osmolite HN, Ross, Ohio, USA) containing carbohydrate (17 g/100 ml), protein (5.3 g/100 ml), and fat (4.1 g/100 ml) was made by mixing the polymeric dietary formula with the artificial gastric juice solution at predetermined volumes resulting in a 10% (v/v) solution. The marker solution containing PEG3350 was made by dissolving 9.438 g (approx) of PEG3350 in 30 ml water, to form a 31.5% solution (approximately) as for Example 7.

The concentration of soluble solids in the artificial gastric juice containing polymeric dietary formulation was measured using the ATAGO® SMART-1 device to obtain pre-dilution concentrations of soluble solids (C1). Next, 30 ml of the 31.5% PEG3350 marker solution was added and mixed with each sample of the artificial gastric juice/polymeric dietary formulation to form post-dilution mixtures. A small sample (e.g. one or two drops) of each of the mixtures of PEG3350 in artificial gastric juice/polymeric dietary formula was then taken for measurement of the soluble solids in the post-dilution samples (C2), using the same refractometer. As before, all measurements were taken at 21° C. Finally, the total volume of solution in the sucrose-human gastric juice mixture was calculated using Equation A.

### Results

The results of this experiment are shown in Table 15. As demonstrated, the soluble solids concentration in the artificial gastric juice samples in 10% Osmolite was higher (i.e. 3.3%) than in Example 5, due to the soluble solids contribution of the polymeric dietary formula. As before, the calculated gastric residual volumes (GRV), based on the pre- and post-dilution soluble solids concentrations (C1 and C2), compared well with the actual volumes (“Total volume”), in all cases being within 10% of the actual volume. These results demonstrate that the method of the invention can be used to accurately calculate gastric residual volumes (GRV) of gastric contents that include dietary formula, by adding a marker solution containing PEG (e.g. PEG3350), and taking measurements of soluble solids concentrations in relatively small samples of the solutions/mixtures.

TABLE 16
Calculated GRV based on artificial gastric juice in 10% dietary
supplement and PEG3350-based marker solution

| Artificial | | | | | Conc. | | | |
|---|---|---|---|---|---|---|---|---|
| gastric | Marker | | Conc. of | Conc. | of | | | |
| juice | solution | Total | marker | of first | second | | Volume | |
| volume, | volume, | volume, | solution, | sample, | sample, | Calculated | difference, | |
| V1 | Vm | V2 | Cm | C1 | C2 | GRV | GRV − V2 | % |
| (ml) | (ml) | (ml) | (%) | (%) | (%) | (ml) | (ml) | difference |
| 20 | 30 | 50 | 31.46 | 3.3 | 20.62 | 48.8 | −1.2 | −2.4% |
| 40 | 30 | 70 | 31.46 | 3.3 | 15.86 | 67.3 | −2.7 | −3.9% |
| 60 | 30 | 90 | 31.46 | 3.3 | 14.57 | 82.3 | −7.7 | −8.6% |
| 80 | 30 | 110 | 30.43 | 3.3 | 11.13 | 103.9 | −6.1 | −5.5% |

The results of Examples 5 to 8 demonstrate how refractometry may be used to calculate the gastric residual volume (GRV) of a subject (e.g. a patient) by mixing (or “diluting”) the gastric contents with a solution having a relatively high concentration of soluble solids, so as to cause an increase in the concentration of soluble solids in the stomach of the subject. The method has particular utility in assessing gastric residual volumes (GRV) in fasting individuals, whom may have a relatively low concentration of soluble solids in their gastric contents. The method also has utility where a subject has received a dietary formula, such as for patients receiving nasogastric feeding. The measurement of gastric residual volumes (GRV) in subjects whom have fasted can be particularly inaccurate using the traditional methods of aspirating the entire gastric contents and measuring the volume of the gastric contents, because the difficulties and limitations of the complete aspiration methods are exacerbated where the gastric residual volume (GRV) is relatively small. In contrast, the method of the invention only requires the aspiration of a sample (i.e. a portion) of the gastric contents, because the devices for measuring soluble solids can operate using only a few drops of solution. Thus, the aspirated sample can be only a tiny proportion (e.g. less than 1 or 2%) of the total gastric residual volume (GRV), and still provide an accurate measurement of gastric residual volume. The method of the invention is not limited to the measurement of soluble solids concentration (e.g. Brix value) by use of a refractometer. Any device for measuring soluble solids can be used, for example, a device that measures soluble solids on the basis of combined refractometry and reflectometry measurements, or other suitable method.

Throughout this specification, unless the context requires otherwise, the word “comprise” or variation such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present application before the priority date of each claim of this application.

It will be appreciated by persons skilled in the art that numerous variations and/or modifications may be made to the embodiments without departing from the spirit or scope of the claims as broadly described. Equivalents for the particular embodiments discussed in this description may practice the claims as well. The present embodiments are, therefore, to be considered in all respects as illustrative and not restrictive.

Further aspects and embodiments of the invention are described as follows.

In one aspect, the invention provides methods of determining the concentration of dietary formula, which, in one embodiment comprises infusing dietary formula into the stomach of a subject, measuring a Brix value of the infused dietary formula, determining a slope value derived from Brix values of serially diluted dietary formula over a determined concentration range, and dividing the Brix value of the infused dietary formula by the slope value (e.g. 0.24). The dietary formula can be, for example, a formula for enteral nutrition, a formula for gastric tube feeding, liquid dietary formula, a polymeric diet formula, etc. In some embodiments, the concentration of dietary formula exhibits a linear relationship to the Brix value of the infused dietary formula. The serially diluted dietary formula can be diluted, for example, in distilled water, gastric juice, saliva, dextrose solution, sodium chloride solution, or other suitable.

In another aspect, the invention provides methods of determining gastric residual volume, which, in one embodiment, comprises (a) measuring the Brix value of a gastric content sample to obtain a pre-dilution Brix value; (b) adding a known volume of water to the gastric content sample to form a post-dilution sample; (c) infusing the post-dilution sample into the stomach of a subject; (d) aspirating the post-dilution sample from the stomach; (e) measuring the Brix value of the aspirated post-dilution sample to obtain a post-dilution Brix value; and (f) multiplying the Brix value of the gastric content sample by the known volume of water and dividing the resulting product by the difference between pre-dilution Brix value and the post-dilution value. In some embodiments, the gastric content sample of step (a) can be obtained, for example, from an in vitro sample or a sample aspirated from a stomach infused with dietary formula. The gastric content sample can be obtained, for example, by aspirating the sample from the stomach of a patient. One or more Brix values can be measured, for example, by refractometer.

In another aspect, the invention provides methods of measuring the volume of dietary formula remaining in stomach, which, in one embodiment, comprises calculating the gastric residual volume of a subject, and multiplying the calculated gastric residual volume by the percent concentration of dietary formula infused in the stomach. The dietary formula can be, for example, a formula for enteral nutrition, a formula for gastric tube feeding, a liquid dietary formula, a polymeric diet formula, etc. Another embodiment of a method of determining the volume of dietary formula remaining in the stomach, comprises calculating a gastric residual volume of the patient infused with dietary formula; measuring the Brix value of an aspirated sample from the infused stomach; and multiplying the gastric residual volume by the Brix value of the aspirated sample and dividing the resulting product by 0.24.

In a further aspect, the invention provides methods of measuring gastric juice volume in stomach, which, in one embodiment, comprises measuring the volume of dietary formula remaining in the stomach of a subject; calculating or measuring the gastric residual volume; and determining the value derived from the difference between the calculated gastric residual volume and the dietary formula volume remaining in the stomach. In embodiments of the method, the volume of dietary formula remaining in stomach can be calculated by determining the gastric residual volume of a subject and multiplying the gastric residual volume by the percent concentration of dietary formula infused in the stomach. In some embodiments, the gastric residual volume is calculated by (a) measuring the Brix value of a gastric content sample to obtain a pre-dilution Brix value; (b) adding a known volume of water to the gastric content sample to form a post-dilution sample; (c) infusing the post-dilution sample into the stomach of a subject; (d) aspirating the post-dilution sample from the stomach; (e) measuring the Brix value of the aspirated post-dilution sample to obtain a post-dilution Brix value; and (f) multiplying the Brix value of the gastric content sample by the known volume of water and dividing the resulting product by the difference between pre-dilution Brix value and the post-dilution value. The dietary formula can be, for example, a formula for enteral nutrition, a formula for gastric tube feeding, a liquid dietary formula, a polymeric diet formula, etc.

In yet another aspect, the invention provides methods of monitoring gastric content emptying and feeding tolerance in a patient receiving dietary formula, which, in one embodiment, comprises infusing dietary formula into the stomach of a patient; calculating a gastric residual volume; aspirating the gastric contents of the patient; measuring the Brix value of the aspirated gastric contents to obtain a pre-dilution Brix value; calculating the gastric residual volume; reinstilling the gastric contents along with a known volume of water; reaspirating at least a portion of the sample and measuring its Brix value to obtain a post-dilution Brix value; and calculating the volume of contents remaining in stomach by multiplying the known volume of water by the pre-dilution Brix value and dividing the resulting product by the difference between the pre-dilution Brix value and the post-dilution Brix value.

In another aspect, the invention provides methods of monitoring feeding tolerance in a patient receiving enteral nutrition, which, in one embodiment, comprises infusing dietary formula into the stomach of a patient; measuring the aspirated gastric residual volume of an infused patient; evaluating the aspirated gastric residual volume, whereby a lower gastric residual volume value indicates acceptable feeding tolerance and a higher gastric residual volume value indicates additional monitoring; calculating a Brix value ratio obtained by a Brix value dilution test; evaluating the Brix value ratio, whereby a ratio of less than approximately 70% indicates acceptable feeding tolerance and a ratio above approximately 70% indicates additional monitoring; calculating the formula volume remaining in the stomach; and evaluating the volume of formula remaining in the stomach, whereby a formula volume approximately less than the infusion rate indicates acceptable feeding tolerance and a formula volume greater than the infusion rate indicates additional monitoring. In embodiments of the method, the lower gastric residual volume is less than approximately 75 ml and the higher gastric residual volume value is above approximately 75 ml. The gastric residual volume can be calculated, for example, by (a) measuring the Brix value of a gastric content sample to obtain a pre-dilution Brix value; (b) adding a known volume of water to the gastric content sample to form a post-dilution sample; (c) infusing the post-dilution sample into the stomach of a subject; (d) aspirating the post-dilution sample from the stomach; (e) measuring the Brix value of the aspirated post-dilution sample to obtain a post-dilution Brix value; and (f) multiplying the Brix value of the gastric content sample by the known volume of water and dividing the resulting product by the difference between pre-dilution Brix value and the post-dilution value. The Brix value ratio can be calculated, for example, by dividing the pre-dilution Brix value by the post dilution Brix value. The Brix value dilution test can further comprise measuring the Brix value of a gastric content sample to obtain a pre-dilution Brix value; adding a known volume of water to the gastric content sample to form a post-dilution sample; infusing the post-dilution sample into the stomach of a subject; aspirating the post-dilution sample from the stomach; measuring the Brix value of the aspirated post-dilution sample to obtain a post-dilution Brix value; and dividing the post-dilution Brix value by the pre-dilution Brix value to obtain a Brix value ratio. The volume of formula remaining in the stomach can be calculated, for example, by determining the gastric residual volume of a subject and multiplying the gastric residual volume by the percent concentration of dietary formula infused in the stomach. In some embodiments, the volume of formula remaining in the stomach is determined by calculating a gastric residual volume of the patient infused with dietary formula; measuring the Brix value of an aspirated sample from the infused stomach; and multiplying the gastric residual volume by the Brix value of the aspirated sample and dividing the resulting product by 0.24.

In still a further aspect, the invention provides a method for determining a volume of a gastric fluid within a stomach of a subject, wherein the method comprises: removing a portion of said gastric fluid from said stomach to provide a first gastric fluid sample; measuring soluble solids in the first gastric sample to provide a first concentration of soluble solids; delivering a marker solution comprising a known concentration of a soluble solid into said stomach to form a diluted gastric fluid within said stomach, said diluted gastric fluid having a concentration of soluble solids greater than the first concentration of soluble solids; removing a portion of the diluted gastric fluid from said stomach to provide a second gastric fluid sample; measuring the soluble solids in said second gastric sample to provide a second concentration of soluble solids; and determining the volume of the gastric fluid within said stomach based on the first and second concentrations of soluble solids and the amount of soluble solid delivered into the stomach. In one embodiment, the volume of gastric fluid within said stomach is calculated by: determining a first difference between the concentration of soluble solids in the marker solution (Cm) and the concentration of soluble solids in the first gastric sample (C1); determining a second difference between the concentration of soluble solids in the second gastric sample (C2) and the concentration of soluble solids in the first gastric sample; and dividing the first difference by the second difference, and multiplying the quotient by the volume of the marker solution (Vm) infused into the stomach. Suitably, the marker solution comprises water. The marker solution may comprise a soluble solid selected from the group consisting of a carbohydrate, sucralose, polyethylene glycol, or wheat dextrin. The marker solution may, for example, comprise PEG3350 and/or sucrose dissolve in water. Conveniently, the gastric fluid samples are removed through a nasogastric tube. Likewise, the marker solution comprising the soluble solid is conveniently delivered through a nasogastric tube. In order that substantially all of the marker solution (and all of the dissolved soluble solid) is delivered into the stomach, it may be beneficial to flush (into the stomach) the apparatus used to deliver the marker solution (e.g. a nasogastric/feeding tube) with liquid. Suitably the liquid used for flushing is water. Having calculated the volume of gastric fluid in the stomach according to this aspect of the invention, the original volume of fluid in the stomach (i.e. the gastric residual volume before delivering marker solution to the stomach) can be calculated, for example, by subtracting the volume of marker solution (and, where used, the volume of liquid used for flushing), from the calculated volume. It will be appreciated, that where a liquid is used for flushing, the volume of the flushing liquid should be added to the volume of the marker solution delivered and taken into account in calculating the effective concentration of the soluble solid in the marker solution. In suitable embodiments, the first (C1) and second (C2) concentrations of soluble solids are determined by refractometery. In one embodiment of the invention, the first and second gastric fluid samples are removed and measured at a set time intervals.

1. A method for determining a volume of a gastric fluid within a stomach of a subject, the method comprising:
removing a portion of said gastric fluid from said stomach to provide a first gastric fluid sample;
measuring soluble solids in the first gastric sample to provide a first concentration of soluble solids;
delivering a marker solution comprising a known concentration of a soluble solid into said stomach to form a diluted gastric fluid within said stomach, said diluted gastric fluid having a concentration of soluble solids greater than the first concentration of soluble solids;
removing a portion of the diluted gastric fluid from said stomach to provide a second gastric fluid sample;
measuring the soluble solids in said second gastric sample to provide a second concentration of soluble solids; and
determining the volume of the gastric fluid within said stomach based on the first and second concentrations of soluble solids, and the volume and concentration of the marker solution delivered to the stomach.

2. The method of claim 1, wherein determining the volume of gastric fluid within said stomach comprises:
determining a first difference between the concentration of soluble solids in the marker solution (Cm) and the concentration of soluble solids in the first gastric sample (C1);
determining a second difference between the concentration of soluble solids in the second gastric sample (C2) and the concentration of soluble solids in the first gastric sample; and
dividing the first difference by the second difference, and multiplying the quotient by the volume of the marker solution (Vm) infused into the stomach.

3. The method of claim 1, wherein the marker solution comprises water.

4. The method of claim 1, wherein the marker solution comprises a soluble solid selected from the group consisting of a carbohydrate, sucralose, polyethylene glycol, or wheat dextrin.

5. The method of claim 1, wherein the gastric fluid is removed through a nasogastric tube.

6. The method of claim 1, wherein the marker solution comprising the soluble solid is delivered through a nasogastric tube.

7. The method of claim 6, wherein after the marker solution has been delivered to the stomach and before the second gastric fluid sample is removed from said stomach, the nasogastric tube is flushed with a known volume of liquid.

8. The method of claim 1, wherein the first (C1) and second (C2) concentrations of soluble solids are determined by refractometery.

9. The method of claim 1, wherein the first and second gastric fluid samples are removed and measured at a set time interval.
